(19) United States
(12) Patent Application Publication
(10) Pub. No.: US 2022/0293214 A1
(43) Pub. Date: Sep. 15, 2022

(54) METHODS OF ANALYZING GENETIC VARIANTS BASED ON GENETIC MATERIAL

(71) Applicant: Fabric Genomics, Inc., Oakland, CA (US)

(72) Inventor: Mark YANDELL, Salt Lake City, UT (US)

(21) Appl. No.: 17/684,853

(22) Filed: Mar. 2, 2022

Related U.S. Application Data
(63) Continuation of application No. PCT/US2020/049557, filed on Sep. 4, 2020.
(60) Provisional application No. 63/005,991, filed on Apr. 6, 2020.
(60) Provisional application No. 62/896,516, filed on Sep. 5, 2019.

(51) Int. Cl. G16B 20/20; C12Q 1/6869; G16B 15/10; G16B 40/20; G06N 3/12

(52) U.S. Cl. CPC G16B 20/20; C12Q 1/6869; G16B 15/10; G16B 40/20; G06N 3/126; C12Q 2600/156

(57) ABSTRACT

Provided are methods for identifying gene variants associated with a phenotype, for example by inferring and scoring of structural variants from whole-genome or exome data or processing a set of genes against a known set of genes having known variants associated with a set of phenotypes, and optionally determining how likely each of the genes are to cause the phenotype.

### CROSS-REFERENCE

The present application is a continuation of International Application No. PCT/US2020/049557, filed Sep. 4, 2020, which claims the benefit of U.S. Provisional Patent Application No. 62/896,516 filed Sep. 5, 2019, and U.S. Provisional Patent Application No. 63/005,991 filed Apr. 6, 2020, the contents of each being hereby incorporated by reference in their entirety.

### BACKGROUND

Next-generation sequencing of genomes and exomes is now being widely used for clinical diagnoses of Mendelian diseases, for idiopathic disease, and for fast diagnosis for newborns in NICUs. Pressures remain to increase diagnostic rate while reducing the cost of the process. Major cost drivers include the cost of sequencing, the computational complexity of data processing pipelines, and time required for clinical interpretation. Expert interpretation typically consists of iterative filtering coupled with evidence review of candidate variants. This process has remained largely manual, time consuming, and because it requires specialized and qualified personnel, drives a significant part of the cost of the test. The number of clinical scientists specialized on genome analysis and variant interpretation is also limited and is not growing as fast as the plans to use genomes in clinical diagnostics. As genome sequencing scales up in the clinic and precision medicine, such manual curation protocols do not scale.

### BRIEF SUMMARY

In some of many aspects, the present disclosure provides a method (e.g., computer-implemented method) for inferring a structural variant (e.g., structural genetic variant), said method comprising: (a) constructing a set of badges to span an exon, an upstream region, a downstream region, a gene regulatory element, or any combination thereof of one or more genes or transcripts thereof; (b) identifying one or more genetic variants having one or more attributes to a subject (e.g., human subject) and overlapping said set of badges, and using said one or more attributes to determine a ploidy of said one or more genetic variants in said set of badges; and (c) reporting a structural variant by using at least said ploidy determined in (b) to indicate a change in gene or regulatory element dosage caused by said structural variant that deviates from a normal human karyotype. In some instances, said ploidy is based at least in part on an analysis of discrepancy in distributing genetic variant alleles zygosities from expectation for a genomic segment overlapped by said badges. In some instances, said ploidy is based at least in part on an analysis of the distribution of reads harboring alternative alleles for said one or more genetic variants overlapped by said badges. In some instances, said one or more genetic variants are from a whole-genome or whole-exome sequencing of a subject (e.g., human subject). In some instances, said badges are configured to span an exon region of a transcript of a human genome. In some instances, said badges further comprise an upstream or a downstream buffer region associated with said exon region of said transcript. In some instances, said badges further comprise a gene regulatory region. In some instances, said badges represent a DNA sequence coordinate of said exon region of said transcript. In some instances, said analysis of sequencing read depth of said genetic variants comprises comparing said read depth of said one or more genetic variants to said one or more genetic variants or each thereof. In some instances, said ploidy is at least partially based on a frequency of its alleles in human populations of said one or more genetic variants. In some instances, said ploidy is inferred by defining an expectation of zygosity of overlapping genetic variants from a subject (e.g., human subject) by comparing to a frequency of its alleles in human populations of said one or more genetic variants. In some instances, said structural variant is associated with a gene associated with a disease phenotype. In some instances, the method further comprises determining a score inferring severity of an impact of a structural variant on a structure of overlapping genes that can be associated with a disease phenotype. In some instances, said structural variant is determined with a predictive positive value of greater than 95%. In some instances, said genetic variants or said genes or transcripts thereof have been ranked by VAAST, VVP, PHEVOR, pVAAST, SIFT, CAD, ANNOVAR, a burden-test, a sequence conservation scoring method, a machine learning method, or any combination thereof. In some instances, the method further comprises prioritizing a single nucleotide variant (SNV), an insertion and/or deletion (INDEL) (for example less than 50 bp), and/or said structural variant. In some instances, the method further comprises automatically prioritizing compound heterozygous genotypes comprising a single nucleotide variant (SNV) or an insertion and/or deletion (INDEL) (for example less than 50 bp) in trans to a larger structural variant. In some instances, said badges are constructed to span each exon of said transcripts. In some instances, said badges are merged if adjacent in genome coordinates, discrepant to expectation of said human normal karyotype, and/or are of similar inferred ploidy. In some instances, zero structural variant is reported. In some instances, at least one structural variant is reported. In some instances, said structural variant is associated with a disease phenotype. In some instances, said structural variant is reported when said badges with said ploidy are greater or less than 44 for autosomes, greater or less than 2 for the X chromosome in females, or greater or less than 1 for the X chromosome in males, or any combination thereof.

In some aspects, the present disclosure provides a method (e.g., computer-implemented method) for inferring a structural variant (e.g., structural genetic variant), said method comprising: (a) providing one or more genetic variants attributable to a subject (e.g., human subject); (b) identifying said one or more genetic variants overlapping one or more badges, wherein said one or more badges represents a portion of a human genome; (c) determining a ploidy of said one or more genetic variants based at least in part on an analysis of sequencing read depth of said one or more genetic variants; and (d) reporting a structural variant by using at least said ploidy determined in (b) to indicate a change in gene or regulatory element dosage caused by said structural variant that deviates from a normal human karyotype. In some instances, said ploidy is based at least in part on an analysis of discrepancy in distributing genetic variant alleles zygosities from expectation for a genomic segment overlapped by said badges. In some instances, said ploidy is based at least in part on an analysis of the distribution of reads harboring alternative alleles for said one or more genetic variants overlapped by said badges. In some instances, said one or more genetic variants are from a whole-genome or whole-exome sequencing of a subject (e.g., human subject). In some instances, said badges are configured to span an exon region of a transcript of a human genome. In some instances, said badges further comprise an upstream or a downstream buffer region associated with said exon region of said transcript. In some instances, said badges further comprise a gene regulatory region. In some instances, said badges represent a DNA sequence coordinate of said exon region of said transcript. In some instances, said analysis of sequencing read depth of said genetic variants comprises comparing said read depth of said one or more genetic variants to said one or more genetic variants or each thereof. In some instances, said ploidy is at least partially based on a frequency of its alleles in human populations of said one or more genetic variants. In some instances, said ploidy is inferred by defining an expectation of zygosity of overlapping genetic variants from a subject (e.g., human subject) by comparing to a frequency of its alleles in human populations of said one or more genetic variants. In some instances, said structural variant is associated with a gene associated with a disease phenotype. In some instances, the method further comprises determining a score inferring severity of an impact of a structural variant on a structure of overlapping genes that can be associated with a disease phenotype. In some instances, said structural variant is determined with a predictive positive value of greater than 95%. In some instances, said genetic variants or said genes or transcripts thereof have been ranked by VAAST, VVP, PHEVOR, pVAAST, SIFT, CAD, ANNOVAR, a burden-test, a sequence conservation scoring method, a machine learning method, or any combination thereof. In some instances, the method further comprises automatically prioritizing compound heterozygous genotypes comprising a single nucleotide variant (SNV) or an insertion and/or deletion (INDEL) (for example less than 50 bp) in trans to a larger structural variant. In some instances, the method further comprises prioritizing said SNV, INDEL, and/or structural variant. In some instances, said badges are constructed to span each exon of said transcripts. In some instances, said badges are merged if adjacent in genome coordinates, discrepant to expectation of said human normal karyotype, and/or are of similar inferred ploidy. In some instances, zero structural variant is reported. In some instances, at least one structural variant is reported. In some instances, said structural variant is associated with a disease phenotype. In some instances, said structural variant is reported when said badges with said ploidy are greater or less than 44 for autosomes, greater or less than 2 for the X chromosome in females, or greater or less than 1 for the X chromosome in males, or any combination thereof.

A method (e.g., computer-implemented method) for analyzing a genome of a subject (e.g., human subject), comprising (a) providing a set of genes or gene regulatory elements suspected of having one or more single nucleotide variants (SNVs), insertions and/or deletions (INDELs), structural variants (e.g., structural genetic variant), or any combination thereof, which set of genes or gene regulatory elements is generated from a nucleic acid sample of said subject; (b) processing said set of genes or gene regulatory elements against a known set of genes or gene regulatory elements to identify at least one gene or gene regulatory element of said set of genes or gene regulatory elements that is associated with a known set of phenotypes, wherein said known set of phenotypes is associated with one or more phenotypes of said subject; (c) calculating a likelihood that said at least one gene or gene regulatory element identified in (b) is causative of a phenotype; and (d) outputting a report that is indicative of said at least one gene or gene regulatory element identified in (b) and said likelihood calculated in (c). In some instances, said set of genes or gene regulatory elements have genetic variants. In some instances, (a) comprises sequencing a DNA sample of said subject. In some instances, said DNA sample comprises a germline DNA sample. In some instances, said DNA sample comprises a somatic tissue DNA sample. In some instances, (b) comprises annotating variant impact on overlapping gene structure or regulatory element locations. In some instances, (b) comprises calculating score variant deleteriousness on gene function. In some instances, (b) comprises gene burden scoring. In some instances, (b) comprises phenotype-based prioritizing. In some instances, said phenotype-based prioritization comprises assigning badges to structural variants identified by another method. In some instances, phenotype-based prioritization is performed without structural variant identified by another method. In some instances, said one or more phenotypes of said subject is the same as one or more phenotypes in said known set of phenotypes. In some instances, said at least one gene or gene regulatory element identified in (b) is causative of a disease, and said disease is associated with said known set of phenotypes. In some instances, (c) comprises calculating a Bayes factor. In some instances, (c) comprises using a machine learning algorithm. In some instances, (c) comprises integrating variant, pedigree, phenotype information, or any combination thereof about said subject. In some instances, (c) comprises using ExAC, gnomAD, OMIM, ClinVar, GARD, Orphanet, HGMD, or any combination thereof as an input source. In some instances, the method further comprises making a diagnostic conclusion based on said report. In some instances, said INDEL is less than 50 bp, or said structural variant is 50 bp or greater than 50 bp.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a flow chart depicting a sample method for identifying genetic variants from a DNA sample of a subject.

FIG. 2 illustrates an extended version of a method for identifying genetic variants from a DNA sample of a subject, further comprising optional steps.

FIG. 3 illustrates a sample calculation of the Bayes factor.

FIG. 4 illustrates a sample implementation of key calculation of a method, including specific methods which can be used for each step, inputs, and data sources.

FIG. 5 illustrates a sample output of data from a method as a report comprising annotations and link-outs to original clinical data sources.

FIG. 6 shows a computer system that is programmed or otherwise configured to implement methods provided herein.

FIG. 7 illustrates a workflow for badge identification and ploidy assessment of a transcript. The example shows a partial deletion encompassing exome 2-6 of a hypothetical transcript. The top panel shows the read coverage density from WGS data derived from read alignments, but not used in this method.

FIG. 8 illustrates merging of adjacent badge groups by ploidy matching.

FIG. 9 illustrates computation of Bayes factors for each gene harbored by a badge group to assess pathogenicity.

FIG. 10 illustrates structural variant calls rescoring by alignment to badge groups.

FIG. 11 shows a table of structural variant pathogenicity assessment for a case with a diagnosis of Prader-Willi Syndrome.

FIG. 12 shows a table depicting a benchmark of the ability to rank causal structural variants in 13 retrospective cases.

### DETAILED DESCRIPTION

Disclosed herein are methods for inference and scoring of structural variants, for example from whole-genome or exome or other genome sequence data, which can significantly improve accuracy and rates of disease diagnosis and benefit personalized medicine and therapeutic treatment. In some instances, the method disclosed herein can improve identification and utilization of structural variants (SVs) in diagnostic process and thus unlock a significant improvement in diagnosis rates, for example for Mendelian conditions and other single-gene diseases. In some instances, SVs disclosed herein can comprise large portions of single genes, entire genes, or several genes at one, effectively either deleting one copy of the normal diploid state resulting in decreased production of proteins, or creating duplications that increase the dosage of their gene product in cells. In some instances, SVs can be a significant contributor of pathogenic genetic variants in Mendelian conditions and other common, complex diseases.

In some instances, the method disclosed herein can be combined with another algorithm to identify candidate disease genes that are affected by variants associated with a set of patient phenotypes. In some instances, the method disclosed herein can comprise combining SV inference and prioritization based on: variant deleteriousness, gene prior derived from patient phenotypes, mining the HPO graph (e.g., Phevor algorithm), prior clinical knowledge from publication databases (e.g., OMIM, CLinVar), or any combination thereof. In some instances, the method disclosed herein can provide zero or substantially free from false positive SVs and/or can identify causal SVs.

In some instances, the method disclosed herein can include possibility of including structural variants as inputs and as part of the assessment with respect to the role of structural variants (SVs) as candidate disease causing variants. In some instances, the method herein comprises scoring large SVs based on the genes overlapped by structural variants. The method herein is superior to the conventional methods which interpret such variants relies on comparing a patient event with databases of events in other patients with similar phenotypes and at best identify the region in common across patients as putatively causative. In some instances, the method disclosed herein can rely on databases of large SVs of congenital syndromes based on microarray data, and identify if the patient event overlaps in a meaningful way with some entries in the database and phenotype match. In some instances, the method disclosed herein can comprise comparing the genes in an event with a database of gene dosage sensitive genes.

In some instances, the method disclosed herein can comprise phenotype prioritization of genes overlapped by the SV as provided by the Phevor algorithm. In some instances, a new SV deleteriousness prior is used instead of VAAST burden test (and which can use gene dosage information), and Phevor output is renormalized in a different way. In some instances, to reduce false positive rate of SV calls from short-read sequencing data, the method herein performs assessment of quality of the provided SV calls by analyzing the entire SV datas from the patient's genome. In some instances, the method disclosed herein produces a clean output and identification of causal SVs through the genes harbored by it, and provides insight on causality of such genes unlike any other conventional approach.

In some instances, the method disclosed herein can identify large SV events from small variant data in the absence of provided SV calls, which can be used to score SVs in genome variants data when SV calls have not been made. In some instances, even when SV calls have been provided, this independent inference of SVs can be used to validate and assess the quality of provided SV calls through alignments between them. In some instances, the method herein can provide increased power to detect causative variants in new cases, and/or utility to re-analyze older cases where SV calls have not been made. In some instances, it has been shown that re-analysis of older cases with the method(s) disclosed herein can yield about 10% or more new diagnoses.

In some instances, the methods disclosed herein can be used in combination to diagnose diseases, for example Mendelian disease, or be used independently for example as a filter for SV calls provided by upstream SV identification algorithms, or in tandem, to downgrade and filter out low quality SV calls.

In some of many aspects, the present disclosure provides a method (e.g., computer-implemented method) for inferring a structural variant, said method comprising: (a) constructing a set of badges to span an exon, an upstream region, a downstream region, or any combination thereof of one or more genes or transcripts thereof; (b) identifying one or more genome variants attributable to a subject (e.g., human subject) and overlapping said set of badges, to determine a ploidy of said one or more genome variants in said set of badges; and (c) using at least said ploidy determined in (b) to indicate a number of structural variants. In some instances, said one or more genome variants are from a genome or exome of a subject (e.g., human subject). In some instances, said badges are configured to span an exon region of a transcript of a human genome. In some instances, said badges further comprise an upstream or a downstream buffer region associated with said exon region of said transcript. In some instances, said badges represent a DNA sequence coordinate of said exon region of said transcript. In some instances, said analysis of a read depth of said genome variants comprises comparing said read depth of said one or more genome variants to each variant in said set of genome variants attributable to a subject (e.g., human subject). In some instances, determining a ploidy of said one or more genome variants is at least partially based on an allele frequency of said one or more genome variants. In some instances, structural variant is associated with a gene associated with a disease phenotype. In some instances, the method further comprises determining a severity score for a structural variant associated with a disease phenotype. In some instances, said one or more structural variants is determined with a predictive positive value of greater than 95%. In some instances, said genome variants or said genes or transcripts thereof have been ranked by VAAST, PHEVOR, pVAAST, SIFT, ANNOVAR, a burden-test, or a sequence conservation scoring method. In some instances, the method further comprises automatically prioritizing compound heterozygous genotypes comprising an SNV or short INDEL in trans to a larger structural variant. In some instances, the method further comprises prioritizing SNV, INDEL, and structural variant. In some instances, said badges are constructed to span each exon of said transcripts. In some instances, said number of structural variants is zero (0). In some instances, said number of structural variants is 1 or great than 1. In some instances, said structural variant is associated with a disease phenotype.

In some aspects, the present disclosure provides a method (e.g., computer-implemented method) for inferring a structural variant, said method comprising: (a) providing a set of genome variants attributable to a subject (e.g., human subject); (b) identifying one or more of set of said genome variants overlapping one or more badges, wherein said one or more badges represents a portion of a human genome; (c) determining a ploidy of said one or more genome variants based at least in part on an analysis of a read depth of said one or more genome variants; and (d) using at least said ploidy determined in (b) to indicate a number of structural variants. In some instances, said one or more genome variants are from a genome or exome of a subject (e.g., human subject). In some instances, said badges are configured to span an exon region of a transcript of a human genome. In some instances, said badges further comprise an upstream or a downstream buffer region associated with said exon region of said transcript. In some instances, said badges represent a DNA sequence coordinate of said exon region of said transcript. In some instances, said analysis of a read depth of said genome variants comprises comparing said read depth of said one or more genome variants to each variant in said set of genome variants attributable to a subject (e.g., human subject). In some instances, determining a ploidy of said one or more genome variants is at least partially based on an allele frequency of said one or more genome variants. In some instances, structural variant is associated with a gene associated with a disease phenotype. In some instances, the method further comprises determining a severity score for a structural variant associated with a disease phenotype. In some instances, said one or more structural variants is determined with a predictive positive value of greater than 95%. In some instances, said genome variants or said genes or transcripts thereof have been ranked by VAAST, PHEVOR, pVAAST, SIFT, ANNOVAR, a burden-test, or a sequence conservation scoring method. In some instances, the method further comprises automatically prioritizing compound heterozygous genotypes comprising an SNV or short INDEL in trans to a larger structural variant. In some instances, the method further comprises prioritizing SNV, INDEL, and structural variant. In some instances, said badges are constructed to span each exon of said transcripts. In some instances, said number of structural variants is zero (0). In some instances, said number of structural variants is 1 or great than 1. In some instances, said structural variant is associated with a disease phenotype.

In some aspects, the present disclosure provides a method for analyzing a genome of a subject, comprising (a) providing a set of genes suspected of having structural variants, which set of genes is generated from a nucleic acid sample of said subject; (b) processing said set of genes against a known set of genes to identify at least one gene of said set of genes that is associated with a known set of phenotypes, wherein said known set of phenotypes is associated with one or more phenotypes of said subject; (c) calculating a likelihood that said at least one gene identified in (b) is causative of a phenotype; and (d) outputting a report that is indicative of said at least one gene identified in (b) and said likelihood calculated in (c). In some instances, said set of genes have variants. In some instances, (a) comprises sequencing a DNA sample of said subject. In some instances, said DNA sample comprises a germline DNA sample. In some instances, (b) comprises annotating variant impact. In some instances, (b) comprises calculating score variant deleteriousness. In some instances, (b) comprises gene burden scoring. In some instances, (b) comprises phenotype-based prioritizing. In some instances, said phenotype-based prioritization comprises assigning badges to structural variants. In some instances, said phenotype-based prioritization is performed without structural variant calls. In some instances, said one or more phenotypes of said subject is the same as one or more phenotypes in said known set of phenotypes. In some instances, said at least one gene identified in (b) is causative of a disease, and wherein said disease is associated with said known set of phenotypes. In some instances, (c) comprises calculating a Bayes factor. In some instances, (c) comprises using a machine learning algorithm. In some instances, (c) comprises integrating variant, pedigree, and phenotype information about said subject. In some instances, (c) comprises using gnomAD, OMIM, and ClinVar as input sources. In some instances, the method further comprises making a diagnostic decision based on said report.

Also provided herein are methods for analyzing a genome of a subject, comprising (a) providing a set of genes having or suspected of having variants, which set of genes is generated from a nucleic acid sample of said subject; (b) processing said set of genes against a known set of genes to identify at least one gene of said set of genes that is associated with a known set of phenotypes, wherein the known set of phenotypes are associated with one or more phenotypes of said subject; (c) calculating a likelihood that said at least one gene identified in (b) is causative of a phenotype; and (d) outputting a report that is indicative of said at least one gene having said variant identified in (b) and said likelihood calculated in (c). In some instances, (a) comprises sequencing a DNA sample of said subject. In some instances, the DNA sample comprises a germline DNA sample. In some instances, (b) comprises variant impact annotation. In some instances, (b) comprises calculation of score variant deleteriousness. In some instances, (b) comprises gene burden scoring. In some instances, (b) comprises phenotype-based prioritization. In some instances, the phenotype based prioritization comprises assigning badges to structural variants. In some instances, the phenotype-based prioritization is performed without structural variant calls. In some instances, the one or more phenotypes of said subject are the same as one or more phenotypes in the known set of phenotypes. In some instances, said at least one gene identified in (b) is causative of a disease, wherein the disease is associated with said known set of phenotypes. In some instances, (c) comprises calculation of a Bayes factor. In some instances, (c) comprises a machine learning algorithm. In some instances, (c) comprises integrating variant, pedigree, and phenotype information about said subject. In some instances, (c) comprises using a genome evaluation database, for example gnomAD, OMIM, and ClinVar as input sources. In some instances, the method further comprises making a diagnostic decision based on the report.

Also provided herein are methods for inferring structural variants, the method comprising: (a) providing a list of transcripts for a list of genes; (b) constructing a set of badges to span each exon of each transcript plus upstream and downstream buffer regions; (c) identifying one or more variants overlapping the badges of a transcript and determine the ploidy of the variants; and (d) identifying one or more structural variants associated with the disease associated with the ploidy. In some instances, there are no structural variant calls in the input. In some instances, the method produces less noise (e.g., signal to noise ratio, or baseline noise) than in a non-badge based method, for example at least about: 10%, 20%, 30%, 40%, or 50% less. In some instances, the method further comprises automatically prioritizes (e.g., computer-operated, not manually), compound heterozygous genotypes comprising a SNV or short INDEL in trans to a larger structural variant. In some instances, the method further comprises prioritizing SNVs, INDELs, and structural variants. In some instances, the set of badges is constructed to span each exon of the transcripts. In some instances, the one or more structural variants is associated with a disease phenotype.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

The details of one or more inventive instances/embodiments are set forth in the accompanying drawings, the claims, and the description herein. Other instances, features, objects, and advantages of the inventive instances/embodiments disclosed and contemplated herein can be combined with any other instance unless explicitly excluded.

Unless otherwise indicated, open terms for example “contain,” “containing,” “include,” “including,” and the like mean comprising.

The terms “a”, “an”, and “the” are used herein to include singular or plural references unless the context clearly dictates otherwise.

Unless otherwise indicated, some instances herein contemplate numerical ranges. When a numerical range is provided, unless otherwise indicated, the range includes the range endpoints. Unless otherwise indicated, numerical ranges include all values and subranges therein as if explicitly written out. Unless otherwise indicated, any numerical ranges and/or values herein can be at 90-110% of the numerical ranges and/or values. For example, the terms “about” in reference to a number or range of numbers is understood to mean the stated number and numbers+/−10% thereof, or 10% below the lower listed limit and 10% above the higher listed limit for the values listed for a range.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “subject” as used herein can refer to a mammal (e.g., a human, mouse, rat, guinea pig, dog, cat, horse, cow, pig, or non-human primate, such as a monkey, chimpanzee or baboon). In some instances, the subject is a human subject. In some instances, the subject is a human subject. In some instances, the subject is a healthy human subject. In some instances, the subject is a human having or suspected of having a genetic disorder or condition.

“Nucleic acid” and “polynucleotide” can be used interchangeably herein, and refer to both RNA and DNA, including cDNA, genomic DNA, synthetic DNA, and DNA or RNA containing nucleic acid analogs. Polynucleotides can have any three-dimensional structure. A nucleic acid can be double-stranded or single-stranded (e.g., a sense strand or an antisense strand). Non-limiting examples of polynucleotides include chromosomes, chromosome fragments, genes, intergenic regions, gene fragments, exons, introns, messenger RNA (mRNA), transfer RNA, ribosomal RNA, siRNA, micro-RNA, ribozymes, cDNA, recombinant polynucleotides, branched polynucleotides, nucleic acid probes and nucleic acid primers. A polynucleotide may contain unconventional or modified nucleotides.

“Nucleotides” are molecules that when joined together for the structural basis of polynucleotides, e.g., ribonucleic acids (RNA) and deoxyribonucleic acids (DNA). A “nucleotide sequence” is the sequence of nucleotides in a given polynucleotide. A nucleotide sequence can also be the complete or partial sequence of an individual's genome and can therefore encompass the sequence of multiple, physically distinct polynucleotides (e.g., chromosomes).

An “individual” can be of any species of interest that comprises genetic information. The individual can be a eukaryote, a prokaryote, or a virus. The individual can be an animal or a plant. The individual can be a human or non-human animal.

The “genome” of an individual member of a species can comprise that individual's partial or complete set of chromosomes, including both coding and non-coding regions. Particular locations within the genome of a species are referred to as “loci”, “sites” or “features”. “Alleles” are varying forms of the genomic DNA located at a given site. In the case of a site where there are two distinct alleles in a species, referred to as “A” and “B”, each individual member of the species can have one of four possible combinations: AA; AB; BA; and BB. The first allele of each pair is inherited from one parent, and the second from the other.

The “genotype” of an individual at a specific site in the individual's genome refers to the specific combination of alleles that the individual has inherited. A “genetic profile” for an individual includes information about the individual's genotype at a collection of sites in the individual's genome. As such, a genetic profile is comprised of a set of data points, where each data point is the genotype of the individual at a particular site.

Genotype combinations with identical alleles (e.g., AA and BB) at a given site are referred to as “homozygous”; genotype combinations with different alleles (e.g., AB and BA) at that site are referred to as “heterozygous.” It has to be noted that in determining the allele in a genome using standard techniques AB and BA cannot be differentiated, meaning it is impossible to determine from which parent a certain allele was inherited, given solely the genomic information of the individual tested. Moreover, variant AB parents can pass either variant A or variant B to their children. While such parents may not have a predisposition to develop a disease, their children may. For example, two variant AB parents can have children who are variant AA, variant AB, or variant BB. For example, one of the two homozygotic combinations in this set of three variant combinations may be associated with a disease. Having advance knowledge of this possibility can allow potential parents to make the best possible decisions about their children's health.

An individual's genotype can include haplotype information. A “haplotype” is a combination of alleles that are inherited or transmitted together. “Phased genotypes” or “phased datasets” provide sequence information along a given chromosome and can be used to provide haplotype information.

The “phenotype” of an individual refers to one or more characteristics. An individual's phenotype can be driven by constituent proteins in the individual's “proteome”, which is the collection of all proteins produced by the cells comprising the individual and coded for in the individual's genome. The proteome can also be defined as the collection of all proteins expressed in a given cell type within an individual. A disease or disease-state can be a phenotype and can therefore be associated with the collection of atoms, molecules, macromolecules, cells, tissues, organs, structures, fluids, metabolic, respiratory, pulmonary, neurological, reproductive or other physiological function, reflexes, behaviors and other physical characteristics observable in the individual through various means.

In many cases, a given phenotype can be associated with a specific genotype. For example, an individual with a certain pair of alleles for the gene that encodes for a particular lipoprotein associated with lipid transport may exhibit a phenotype characterized by a susceptibility to a hyperlipidemous disorder that leads to heart disease.

The “background” or “background database” can be a collection of nucleotide sequences (e.g., one or more genes or gene fragments, one or more chromosomes or chromosome fragments, one or more genomes or genome fragments, one or more transcriptome sequences, etc.) and their variants (variant files) used to derive reference variant frequencies in the background sequences. The background database can contain any number of nucleotide sequences and can vary based upon the number of available sequences. The background database can contain about 1-10000, 1-5000, 1-2500, 1-1000, 1-500, 1-100, 1-50, 1-10, 10-10000, 10-5000, 10-2500, 10-1000, 10-500, 10-100, 10-50, 50-10000, 50-5000, 50-2500, 50-1000, 50-500, 50-100, 100-10000, 100-5000, 100-2500, 100-1000, 100-500, 500-10000, 500-5000, 500-2500, 500-1000, 1000-10000, 1000-5000, 1000-2500, 2500-10000, 2500-5000, or 5000-10000 sequences, or any included sub-range; for example, about 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 600, 700, 800, 900, 1000, 1250, 1500, 1750, 2000, 2500, 3000, 3500, 4000, 4500, 5000, 6000, 7000, 8000, 9000, 10000, or more sequences, or any intervening integer.

The “target” or “case” can be a collection of nucleotide sequences (e.g., one or more genes or gene fragments, one or more genomes or genome fragments, one or more transcriptome sequences, etc.) and their variants under study. The target can contain information from individuals that exhibit the phenotype under study. The target can be an individual genome sequence or collection of individual genome sequences. The personal genome sequence can be from an individual diagnosed with, suspected of having, or at increased risk for a disease. The target can be a tumor genome sequence. The target can be genetic sequences from plants or other species that have desirable characteristics.

The term “cohort” can be used to describe a collection of target or background sequences, and their variants, used in a given comparison. A cohort can include about 1-10000, 1-5000, 1-2500, 1-1000, 1-500, 1-100, 1-50, 1-10, 10-10000, 10-5000, 10-2500, 10-1000, 10-500, 10-100, 10-50, 50-10000, 50-5000, 50-2500, 50-1000, 50-500, 50-100, 100-10000, 100-5000, 100-2500, 100-1000, 100-500, 500-10000, 500-5000, 500-2500, 500-1000, 1000-10000, 1000-5000, 1000-2500, 2500-10000, 2500-5000, or 5000-10000 sequences, or any included sub-range; for example, about 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 600, 700, 800, 900, 1000, 1250, 1500, 1750, 2000, 2500, 3000, 3500, 4000, 4500, 5000, 6000, 7000, 8000, 9000, 10000, or more sequences, or any intervening integer.

A “variant” can be any change in an individual nucleotide sequence compared to a reference sequence. The reference sequence can be a single sequence, a cohort of reference sequences, or a consensus sequence derived from a cohort of reference sequences. An individual variant can be a coding variant or a non-coding variant. A variant wherein a single nucleotide within the individual sequence is changed in comparison to the reference sequence can be referred to as a single nucleotide polymorphism (SNP) or a single nucleotide variant (SNV) and these terms are used interchangeably herein. SNPs that occur in the protein coding regions of genes that give rise to the expression of variant or defective proteins are potentially the cause of a genetic-based disease. In some instances, SNPs that occur in non-coding regions can result in altered mRNA and/or protein expression.

Examples can include SNPs that defective splicing at exon/intron junctions. Exons are the regions in genes that contain three-nucleotide codons that are ultimately translated into the amino acids that form proteins. Introns are regions in genes that can be transcribed into pre-messenger RNA but do not code for amino acids. In the process by which genomic DNA is transcribed into messenger RNA, introns are often spliced out of pre-messenger RNA transcripts to yield messenger RNA. A SNP can be in a coding region or a non-coding region. A SNP in a coding region can be a silent mutation, otherwise known as a synonymous mutation, wherein an encoded amino acid is not changed due to the variant. An SNP in a coding region can be a missense mutation, wherein an encoded amino acid is changed due to the variant. An SNP in a coding region can also be a nonsense mutation, wherein the variant introduces a premature stop codon. A variant can include an insertion and/or deletion (INDEL) of one or more nucleotides. An INDEL can be a frame-shift mutation, which can alter a gene product, for example to a significant extent. An INDEL can be a splice-site mutation. A variant can be a large-scale mutation in a chromosome structure; for example, a copy-number variant caused by an amplification or duplication of one or more genes or chromosome regions or a deletion of one or more genes or chromosomal regions; or a translocation causing the interchange of genetic parts from non-homologous chromosomes, an interstitial deletion, or an inversion.

Variants can be provided in a variant file, for example, a genome variant file (GVF) or a variant call format (VCF) file. According to the methods disclosed herein, tools can be provided to convert a variant file provided in one format to another more desired format. A variant file can comprise frequency information on the included variants.

A “feature” can be any span or a collection of spans within a nucleotide sequence (e.g., a genome or transcriptome sequence). A feature can comprise a genome or genome fragment, one or more chromosomes or chromosome fragments, one or more genes or gene fragments, one or more transcripts or transcript fragments, one or more exons or exon fragments, one or more introns or intron fragments, one or more splice sites, one or more regulatory elements (e.g., a promoter, an enhancer, a repressor, etc.) one or more plasmids or plasmid fragments, one or more artificial chromosomes or fragments, or a combination thereof. A feature can be automatically selected. A feature can be user-selectable.

A “disease gene model” can refer to the mode of inheritance for a phenotype. A single gene disorder can be autosomal dominant, autosomal recessive, X-linked dominant, X-linked recessive, Y-linked, or mitochondrial. Diseases can also be multifactorial and/or polygenic or complex, involving more than one variant or damaged gene.

Pedigree” can refer to lineage or genealogical descent of an individual. Pedigree information can include polynucleotide sequence data from a known relative of an individual such as a child, a sibling, a parent, an aunt or uncle, a grandparent, etc.

“Amino acid” or “peptide” refers to one of the twenty biologically occurring amino acids and to synthetic amino acids, including D/L optical isomers. Amino acids can be classified based upon the properties of their side chains as weakly acidic, weakly basic, hydrophilic, or hydrophobic. A “polypeptide” refers to a molecule formed by a sequence of two or more amino acids. Proteins are linear polypeptide chains composed of amino acid building blocks. The linear polypeptide sequence provides only a small part of the structural information that can be important to the biochemist, however. The polypeptide chain folds to give secondary structural units (most commonly alpha helices and beta strands). Secondary structural units can then fold to give supersecondary structures (for example, beta sheets) and a tertiary structure. Most of the behaviors of a protein are determined by its secondary and tertiary structure, including those that can be important for allowing the protein to function in a living system. [0043] Disclosed herein is a newly developed analysis method that can analyze personal genome sequence data. The input of the method can be a genome file. The genome file can comprise genome sequence files, partial genome sequence files, genome variant files (e.g., VCF files, GVF files, etc.), partial genome variant files, genotyping array files, or any other DNA variant files. The genome variant files can contain the variants or difference of an individual genome or a set of genomes compared to a reference genome (e.g., human reference assembly). These variant files can include variants such as single nucleotide variants (SNVs), single nucleotide polymorphisms (SNPs), small and larger, insertion and/or deletions (INDELS), rearrangements, CNV (copy number variants), Structural Variants (SVs), etc. The variant file can include frequency information for each variant.

Described herein are methods for analyzing a genome of a subject. Such methods can comprise providing a set of genes having variants. Such genes can be causative of disease, affected by disease, or suspected of being causative of or affected by disease.

The set of genes can be processed against a known set of genes. This known set of genes can comprise known variants, which can be associated with a set of phenotypes. By processing these genes, one or more genes of the set of genes can be identified as having a variant associated with a phenotype of the set of phenotypes. For example, a variant of a gene can be associated with a disease. A process that can be used for the identification is the Variant Annotation, Analysis, and Selection Tool (VAAST), which is described in the PCT publication WO2012034030. In some cases,

In some cases, where phenotypic descriptions of the patient are available, these can be also be used to prioritize genes with respect to their likelihood to be involved in such phenotypes based on prior knowledge. For example, a likelihood that said gene identified can be causative of said phenotype. In some cases the PHEVOR algorithm can be used in such a step.

In some cases, the method can further comprise outputting a report. Such a report can indicate that at least one gene has a variant identified in (b). In some cases, such a report can indicate that at least one gene having a variant in (b) has a likelihood calculated in (c).

Methods described herein can be applied to a multitude of applications. For example, applications can include diagnostics of rare genetic disease (a.k.a. single gene or Mendelian disease), rapid diagnostics of newborns in the neonatal intensive care unit, rapid diagnostics of children in the pediatric intensive care unit, fast scanning for the presence of monogenic or oligogenic disease variants in cohorts for research studies for the identification of covariates, confounders, comorbidities, or quality control, diagnostics of oligogenic genetic diseases (diseases where more than one gene contributes, each with lower penetrance, e.g. some instances of Chron's Disease), or other applications.

### VAAST

VAAST can be a probabilistic disease-gene finder. VAAST can be an ab initio tool which can identify a new genetic disease using personal genome sequences. Examples of disease genes and disease-causing alleles which have been identified using VAAST include the involvement of NFKB2 in Common Variable Immunodeficiency, the gene and somatic alleles that can cause Sturge-Weber syndrome, a novel role for STAT1 in enteropathy, maternal coding variants, in complement receptor 1 that can cause a predisposition for spontaneous idiopathic preterm birth; a role for RINT1 that can predispose carriers to breast or Lynch Syndrome spectrum cancers; a novel allele in MYH6 that can cause Wolff-Parkinson-White Syndrome; variant(s) causing Primary Ovarian Insufficiency; Autoimmunity which can be due to RAG deficiency; and the role of Retinoic Acid Signaling Pathway can have in Total Anomalous Pulmonary Venous Return (TAPVR). VAAST and its adjunct tools have been employed in a number of non-human studies, demonstrating the robustness of its approach to genotype-phenotype association, and highlighting the power of VAAST to identify non-coding alleles in cis-regulatory regions and interacting genes PHEVOR.

PHEVOR can leverage the Human Phenotype and Gene ontologies for integration of deep phenotype data with genome sequence information. PHEVOR can integrate phenotype, gene function, and disease information with personal genomic data to identify disease-causing alleles. PHEVOR can combine knowledge resident in biomedical ontologies with the output(s) of variant prioritization tool(s). In some cases, PHEVOR can use an algorithm that can propagate information across ontologies, between ontologies, or both. PHEVOR can accurately reprioritize potentially damaging alleles using one or more variant prioritization tools considering knowledge of one or more of gene function knowledge, disease knowledge, or phenotype knowledge. In some cases, PHEVOR can performed single exome analysis or family trio-based analysis.

In some cases, PHEVOR is not limited to known diseases-or known disease-causing alleles. In some cases, PHEVOR can use information in one or more ontologies, including latent information, to discover genes and/or disease-causing alleles that are not previously associated with a disease at the time of analysis.

### Subjects

Subjects described herein can be subjects having genetic material which can be analyzed using methods described herein.

A subject can be a human, an animal, or another organism having a genome. In some cases, a subject can have a disease or be suspected of having a disease. A subject can have one or more symptoms or phenotypes associated with a disease.

A subject can have one or more family members, such that the family members can provide a genetic material sample to be used by the method to identify genes or gene variants in the sample of the subject. Family members can include parents (e.g., mother or father), siblings (e.g., brother or sister), aunts, uncles, cousins, grandparents, great-grandparents, children, or other relatives. A relative can be healthy. In some cases, a relative can have a disease or be suspected of having a disease. In some cases, a relative can have or be suspected of having a same disease that the subject has or is suspected of having.

A subject can be apparently healthy. In some cases, a subject can have or be suspected of having a disease. The disease can be known or unknown. In some cases, the subject can have or be suspecting of having two or more diseases.

A disease can comprise a disease that has a genetic component that can lead to said disease, or a genetic component that can predispose a subject to that disease (i.e., make a subject more likely than others to develop or have the disease). Diseases can comprise

### Methods

Methods described herein can be used to identify a gene variant that can be causative of a phenotype, or a likelihood that a gene variant is causative of a phenotype. Such methods can comprise an integration of outputs of algorithms (e.g., variant prioritizing algorithms such as VAAST and PHEVOR) to identify disease causing genes in the genome of a subject. In some cases, the method can additionally integrate one or more of a proband genotype, one or more parental genotypes, knowledge from the Online Mendelian Inheritance in Man (OMIM) database, knowledge from the Genome Aggregation Database (gnomAD), or knowledge from the ClinVar database to identify disease causing genes in the genome of a subject. In some cases, disease-causing genes in a genome of a patient can be identified quickly, and in some cases the speed can be compatible with urgent clinical cases. Outputs of methods used herein can comprise one or more predicted disease-causing variants, a disease mode, an inheritance mode, and a confidence value.

OMIM is a database that catalogues all the known diseases with a genetic component, and— when possible— links them to the relevant genes in the human genome and provides references for further research and tools for genomic analysis of a catalogued gene (see www.omim.org). OMIM is one of the databases housed in the U.S. National Center for Biotechnology Information (NCBI) and included in its search menus. Every disease and gene is assigned a six digit number of which the first number classifies the method of inheritance. If the initial digit is 1, the trait is deemed autosomal dominant; if 2, autosomal recessive; if 3, X-linked. Wherever a trait defined in this dictionary has a MIM number, the number from the 12th edition of MIM, is given in square brackets with or without an asterisk (asterisks indicate that the mode of inheritance is known; a number symbol (#) before an entry number means that the phenotype can be caused by mutation in any of two or more genes) as appropriate (e.g., Pelizaeus-Merzbacher disease [MIM #312080] is an X-linked recessive disorder).

Range of MIM codes for method of inheritance: 100000-299999: autosomal loci or phenotypes (created before May 15, 1994); 300000-399999: X-linked loci or phenotypes; 400000-499999: Y-linked loci or phenotypes; 500000-599999: Mitochondrial loci or phenotypes; 600000-above: Autosomal loci or phenotypes (created after May 15, 1994). Allelic variants (mutations) are designated by the MIM number of the entry, followed by a decimal point and a unique 4-digit variant number. For example, allelic variants in the factor IX gene (300746) are numbered300746.0001 through 300746.0101.

In some instances, the method of identifying and/or prioritizing phenotype causing variants by comparing a target cohort to a background cohort incorporates Amino Acid Substitution (AAS) information from the OMIM database.

GnomAD is a database developed to aggregate and harmonize exome sequencing data and genome sequencing data. Data in gnomAD can originate from sequencing projects such as large-scale sequencing projects. In some cases, gnomAD can provide summary data for sequencing data. gnomAD can span at least 125,748 exome sequences and at least 15,708 whole-genome sequences. Sequences in gnomAD can be from unrelated individuals. The individuals can be sequenced as part of one or more studies, such as disease-specific genetic studies or population genetic studies.

ClinVar is a freely accessible, public archive of reports of the relationships among human variations and phenotypes, with supporting evidence. In some cases, ClinVar can facilitate access to and communication about the relationships asserted between human variation and observed health status, and in some cases the history of such an interpretation. ClinVar can report variant(s) found in a patient sample, assertions made regarding the clinical significance of a variant, information about the submitter, and other supporting data. In some cases, the alleles can be mapped to reference sequences, and can be reported according to the Human Genome Variation Society standard.

Accessions in ClinVar, for example with the format SCV000000000.0, can be assigned to each submitted record. In some cases, when there are multiple submitted records about the same variation/condition pair, they can be aggregated within ClinVar's data flow and reported as a reference accession with the format RCV000000000.0. Thus, in some cases, one variant can be included in multiple RCV accessions whenever different conditions are reported for that variant. Submitted records for the same variation can be also aggregated and reported as an accession with the format VCV000000000.0. This aggregation can allow a user to review all submitted data for a variant, regardless of the condition for which it was interpreted.

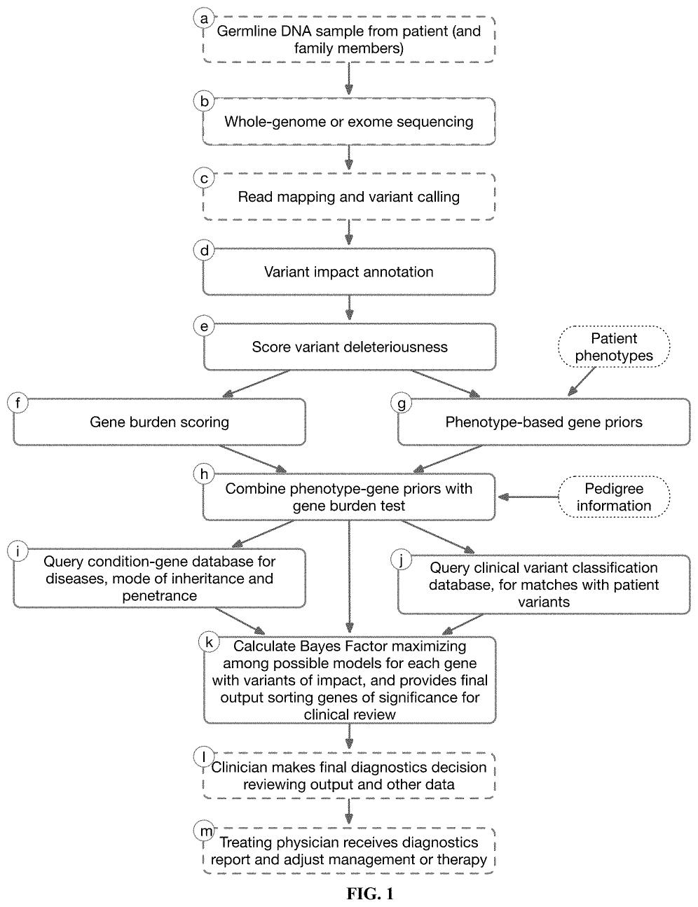

The basic workflow of the method is depicted in FIG. 1. In this figure, method steps which can be core steps are depicted as boxes having solid lines, while steps that can be necessary to the method but are also consistent with the VAAST or PHEVOR method are depicted as boxes having dashed lines. Rounded boxes having small dotted lines are additional metadata inputs which can be used by the method.

Briefly, the method comprises (a) providing a set of genes having variants, which set of genes is generated from a nucleic acid sample of said subject; (b) processing said set of genes against a known set of genes to identify at least one gene of said set of genes having a variant, wherein said known set of genes have known variants associated with a set of phenotype, and wherein said variant is associated with a phenotype of said set of phenotypes; (c) calculating a likelihood that said gene identified in (b) is causative of said phonotype; and (d) outputting a report that is indicative of said at least one gene having said variant identified in (b) and said likelihood calculated in (c). Alternatively, the method described herein may comprise additional steps (e.g., step (e), step (f), step (g), step (h), step (i), step (j), step (k), or step (l)).

The first step (a) of the basic method depicted in FIG. 1 is providing a genetic material sample from a subject. The genetic material sample can be a DNA sample, such as a germline DNA sample. In some cases, another DNA sample such as a somatic DNA sample can be collected. In the next step (b), the sample can be subjected to sequencing, such as by using a nucleotide sequencing method. Sequencing can comprise for example genome sequencing, exome sequencing, or both.

Nucleotide sequencing information can be obtained using any known or future methodology or technology platform; for example, Sanger sequencing, dye-terminator sequencing, Massively Parallel Signature Sequencing (MPSS), Polony sequencing, 454 pyrosequencing, Illumina sequencing, SOLiD sequencing, ion semiconductor sequencing, DNA nanoball sequencing, sequencing by hybridization, or any combination thereof. Sequences from multiple different sequencing platforms can be used in the comparison. Non-limiting examples of types of sequence information that can be utilized in the methods disclosed herein are whole genome sequencing (WGS), exome sequencing, and exon-capture sequencing. The sequencing can be performed on paired-end sequencing libraries.

Sequencing data can be aligned to any known or future reference sequence. For example, if the sequencing data is from a human, the sequencing data can be aligned to a human genome sequence (e.g., any current or future human sequence, e.g., hg19 (GRCh37), hgl 8, hgl7, hgl6, hgl5, hgl3, hgl2, hgl 1, hg8, hg7, hg6, hg5, hg4, etc.). (See hgdownload.cse.ucsc.edu/downloads.html). In some instances, the reference sequence is provided in a Fasta file. Fasta files can be used for providing a copy of the reference genome sequence. Each sequence (e.g., chromosome or a contig) can begin with a header line, which can begin with the ‘>’ character. The first contiguous set of non-whitespace characters after the ‘>’ can be used as the ID of that sequence. In some instances, this ID must match the ‘seqid’ column described supra for the sequence feature and sequence variants. On the next and subsequent lines the sequence can be represented with the characters A, C, G, T, and N. In some instances, all other characters are disallowed. The sequence lines can be of any length. In some instances, all the lines must be the same length, except the final line of each sequence, which can terminate whenever necessary at the end of the sequence.

The GFF3 file format can be used to annotate genomic features in the reference sequence. The GFF3 (General Feature Format version 3) file format is a widely used format for annotating genomic features. Although various versions of GTF and GFF formats have been in use for many years, GFF3 was introduced by Lincoln Stein to standardize the various gene annotation formats to allow better interoperability between genome projects. The Sequence Ontology group currently maintains the GFF3 specification. (See www.sequenceontology.org/resources/gff3.html). Briefly, a GFF3 file can begin with one or more lines of pragma or meta-data information on lines that begin with ‘##’. In some instances, a required pragma is ‘##gff-version 3’. Header lines can be followed by one or more (usually many more) feature lines. In some instances, each feature line describes a single genomic feature. Each feature line can consist of nine tab-delimited columns. Each of the first eight columns can describe details about the feature and its location on the genome and the final line can be a set of tag value pairs that describe attributes of the feature.

A number of programs have been developed to perform sequence alignments and the choice of which particular program to use can depend upon the type of sequencing data and/or the type of alignment that may be required; for example, programs have been developed to perform a database search, conduct a pairwise alignment, perform a multiple sequence alignment, perform a genomics analysis, find a motif, perform benchmarking, and conduct a short sequence alignment. Examples of programs that can be used to perform a database search include BLAST, FASTA, HMMER, IDF, Infernal, Sequilab, SAM, and SSEARCH. Examples of programs that can be used to perform a pairwise alignment include AC ANA, Bioconductor Biostrings::pairwiseAlignment, BioPerl dpAlign, BLASTZ, LASTZ, DNADot, DOTLET, FEAST, JAligner, LALIGN, mAlign, matcher, MCALIGN2, MUMmer, needle, Ngila, PatternHunter, ProbA (also propA), REPuter, Satsuma, SEQALN, SIM, GAP, NAP, LAP, SIM, SPA: Super pairwise alignment, Sequences Studio, SWIFT suit, stretcher, tranalign, UGENE, water, wordmatch, and YASS. Examples of programs that can be used to perform a multiple sequence alignment include ALE, AMAP, anon., BAli-Phy, CHAO S/DI ALIGN, ClustalW, CodonCode Aligner, DIALIGN-TX and DIALIGN-T, DNA alignment, FSA, Geneious, Kalign, MAFFT, MARNA, MAVID, MSA, MULTALIN, Multi-LAGAN, MUSCLE, Opal, Pecan, Phylo, PSAlign, RevTrans, Se-Al, StatAlign, Stemloc, T-Coffee, and UGENE. Examples of programs that can be used for genomics analysis include ACT (Artemis Comparison Tool), AVID, BLAT, GMAP, Mauve, MGA, Mulan, Multiz, PLAST-ncRNA, Sequerome, Sequilab, Shuffle-LAGAN, SIBsim4/Sim4, and SLAM. Examples of programs that can be used for finding motifs include BLOCKS, eMOTIF, Gibbs motif sampler, HMMTOP, I-sites, MEME/MAST, MERCI, PHI-Blast, Phyloscan, and TEIRESIAS. Examples of programs that can be used for benchmarking include BAliBASE, HOMSTRAD, Oxbench, PFAM, PREFAB, SABmark, and SMART. Examples of software that can be used to perform a short sequence alignment include BFAST, BLASTN, BLAT, Bowtie, BWA, CASHX, CUDA-EC, drFAST, ELAND, GNUMAP, GEM, GMAP and GSNAP, Geneious Assembler, LAST, MAQ, mrFAST and mrsFAST, MOM, MOSAIK, MPscan, Novoalign, NextGENe, PALMapper, PerM, QPalma, RazerS, RMAP, rNA, RTG Investigator, Segemehl, SeqMap, Shrec, SHRiMP, SLIDER, SOAP, SOCS, SSAHA and SSAHA2, Stampy, SToRM, Taipan, UGENE, XpressAlign, and ZOOM. In some instances, sequence data is aligned to a reference sequence using Burroughs Wheeler alignment (BWA).

Sequence alignment data can be stored in a SAM file. SAM (Sequence Alignment/Map) is a flexible generic format for storing nucleotide sequence alignment (see samtools.sourceforge.net/SAM1.pdf). Sequence alignment data can be stored in a BAM file, which is a compressed binary version of the SAM format (see genome.ucsc.edu/FAQ/FAQformat.html#format5.1). In some instances, sequence alignment data in SAM format is converted to BAM format.

In some cases, small variants can be identified in the method disclosed herein for example in a step (b). Such variants can comprise for example one or more of a single nucleotide change, a small insertion, or a small deletions. In some cases, where step (b) can identify structural variants. Such variants can comprise for example one or more of a large insertion, a large deletion, a repeat expansion, an inversion, or a translocation.

The method disclosed herein for example next step (c) can comprise read mapping and variant calling.

Read mapping can comprise aligning short reads (e.g., sequences) to a reference sequence. A reference sequence can be a complete genome, a transcriptome, a de novo assembly, or another sequence. Read mapping can output a sequence/alignment map (SAM) for one or more of the samples. The SAM file can indicate one or more of the reference sequence to which the sample maps, the position in the reference, or a scales quality score of the mapping. In some cases, a SAM file can be used to extract gene expression information and/or to identify variants in the sequencing data.

Read mapping can be performed using an algorithm, such as the read mapping and SNV calling algorithm, GNUMAP, which was also independently used to align the reads from the Illumina.qseq files to the X chromosome (human sequence build 36) and to simultaneously call SNVs. GNUMAP utilizes a probabilistic Pair-Hidden Markov Model (PHMM) for base calling and SNV detection that incorporates base uncertainty based on the quality scores from the sequencing run, as well as mapping uncertainty from multiple optimal and sub-optimal alignments of the read to a given location to the genome. In addition, this approach applies a likelihood ratio test that provides researchers with straightforward SNV calling cutoffs based on a p-value cutoff or a false discovery control. Reads were aligned and SNVs called for the five samples. SNV calls for 111-4, brother, and uncle were made assuming a haploid genome (because the calls are on the X chromosome) whereas heterozygous calls were allowed for the mother and grandmother. SNVs were selected based on a p-value cutoff of 0.001. Due to the X-linked nature of the disease, candidate SNVs were selected that are heterozygous in the mother and grandmother, and different between the uncle/brother and 111-4.

Variant calling can be a process by which variants can be identified by sequence data. Variant calling can be performed after sequencing and read mapping. Variant calling can comprise identifying where an aligned read/sequence differs from a reference genome. Variant calling can be somatic variant calling or germline variant calling. In germline variant calling, a reference genome can be standard for the species of interest. This can allow the identification of genotypes. Given a diploid genome, at any given locus, either all reads can have the same base, which can indicate homozygosity, or approximately half of all reads can have one base while the other half has another base, indicating heterozygosity. In some cases, an exception to this rule (e.g., monoploid) can include the sex chromosomes in a male mammal. In somatic variant calling, the reference can be a related tissue from the same individual. In somatic variant calling, mosaicism between cells can be observed.

Variant calling can be error-prone in repetitive and/or homologous sequences. Variant calling errors in repetitive or homologous sequences can be caused by short sequence reads aligning to multiple sites within the reference sequence.

In some instances, a method disclosed herein for example step (d) can comprise variant impact annotation. Variant impact annotation can comprise predicting an effect or function of an individual variant. In some cases, the biological information can be extracted, collected, and/or displayed. Variant impact annotation can comprise gene based annotation, which can comprise using information from a gene as a reference to indicate whether an observed variant resides in or near a gene, and if that variant has the potential to disrupt a protein sequence or the function of a protein. Variant impact annotation can comprise knowledge based annotation, which can be based on information of a gene attribute, function, or metabolism. Knowledge based annotation can emphasize genetic variation that can be disruptive to a protein function domain, protein-protein interaction, or biological pathway. Variant impact annotation can comprise functional annotation, which can identify variant function based on information about whether a variant locus is in a known functional region that can harbor genomic signals or epigenomic signals. Functional annotation can include annotation regarding transcriptional gene regulation, alternative splicing, RNA processing and post-transcriptional regulation, translation and post translational modifications, protein function, or evolutionary conservation and natural selection.

In some instances, a method disclosed herein for example step (e) can comprise scoring variant deleteriousness. Scoring can comprise genome-wide scoring. An example of genome wide scoring can comprise that performed by the Genomic Evolutionary Rate Profiling (GERP) software, for example on multiple sequence alignments of whole-genomes. Algorithms such as GERP can identify constrained loci in multiple sequence alignments by comparing the level of substitution observed to that expected if there was no functional constraint. Another example of a genome wide scoring is the Combined annotation Dependent Depletion (CADD) tool, which can predict deleteriousness of single nucleotide variants and insertion/deletions variants in the human genome by integrating multiple annotations including conservation and functional information into one metric.

Scoring variant deleteriousness can comprise using an algorithm capable of evaluating missense variants. For example, an algorithm such as the SIFT algorithm can predict whether an amino acid substitution is likely to affect protein function based on sequence homology and the physico-chemical similarity between the alternate amino acids. As another example, an algorithm such as PolyPhen algorithms can predict the effect of an amino acid substitution on the structure and function of a protein using sequence homology, Pfam annotations, 3D structures from PDB where available, and a number of other databases and tools (including DSSP, ncoils etc.). As another example, an algorithm such as the Rare Exome Variant Ensemble Lerner (REVEL) can predict the pathogenicity of missense variants by integrating scores from MutPred, FATHMM v2.3, VEST 3.0, PolyPhen-2, SIFT, PROVEAN, MutationAssessor, MutationTaster, LRT, GERP++, SiPhy, phyloP, and phastCons. As another example, an algorithm such as MetaLR can use logistic regression to integrate nine independent variant deleteriousness scores and allele frequency information to predict the deleteriousness of missense variants. In another example, an algorithm such as MutationAssessor can predict the functional impact of amino-acid substitutions in proteins using the evolutionary conservation of the affected amino acid in protein homologs.

In some instances, a method disclosed herein for example (e) can comprise calculating the probability of variant deleteriousness based on a model of their impact in the function and/or expression of the gene product. In some instances, a method disclosed herein for example (e) can comprise calculating by the VAAST Variant Prioritization Score (VVP), As another example, (e) can comprise calculating by the VAAST Variant Prioritization Score (VVP) taking into consideration the allele frequencies from public databases such as gnomAD. In some instances, a method disclosed herein for example (e) can comprise calculating by any variant deleteriousness score, such as SIFT 22, Polyphen 23, CAD, Revel 24, ClinPred 25, primateAl 26, etc. In some instances, a method disclosed herein for example (e) can comprise calculating the probability of variant deleteriousness based on a model of their impact in the function and timing and location of expression of the gene product. In some instances, a method disclosed herein for example (e) can comprise calculating the probability of variant deleteriousness based on a model of their impact in the function and timing and location of expression of the gene product using multi-omics approaches. In some instances, a method disclosed herein for example (e) can comprise calculating the probability of variant deleteriousness based on a model of their impact in the function of the gene product using phylogenetic approaches. In some instances, a method disclosed herein for example (e) can comprise calculating the probability of variant deleteriousness based on a model of their impact in the function of the gene product trained from labeled positive and/or negative examples using machine learning approaches.

In some instances, a method disclosed herein for example (e) can comprise assessing the deleteriousness of a variant by a functional assay. In some instances, a method disclosed herein for example (e) can comprise querying the variants in databases of functional assay results and obtaining a functional value or score for such variant in the assay. In some cases, the assay can comprise screening variants generated in vitro or in vivo by a systematic mutagenic assay. In some cases, the assay can comprise screening variants generated in vitro or in vivo when the systematic mutagenic assay used gene-editing technology.

In some instances, a method disclosed herein for example (e) can comprise phenotype prioritization of genes overlapped by the structural variant. In some instances, for example a new structural variant deleteriousness prior can be used instead of a VAAST burden test or other acceptable method for (e) provided herein.

In some instances, such phenotype prioritization can comprise improvement of a false positive rate. In some instances, a false positive rate of a step or a method can be improved if the number of false positive structural variants is less than when using a different method, or less than predicted. In some instances, reduction of a false positive rate can be reduction of false positive structural variants called from a sequencing data (e.g., short-read sequencing data). In some cases, the method can perform assessment of quality of the provided structural variant calls, for example by analyzing structural variant data from a genome sequence of a patient or a subject. In some cases, such a method can be used to re-analyze a data set where sequence variant calls have not been made. In some instances, such re-analysis can yield a new diagnosis in about 5%, about 10%, or about 15% of cases. In some instances, such re-analysis can yield a new diagnosis in at least 5%, at least 10%, or at least 15% of cases. This structural variance inference and vetting method is further described hereinbelow. A new diagnosis may include predicting a presence of a disease or disorder that was or was not suspected based on a previous analysis of the genome sequence data.

In some instances, a method disclosed herein for example step (e) can be accomplished using a method to discover large structural variant events from small variant data in the absence of provided structural variant calls. In some cases, this can be used to score structural variants in genome variant data when structural variant calls have not been made. In some instances, this independent inference of structural variants can be used to validate and assess the quality of provided structural variant calls, for example through alignment of the structural variants. This validation can be performed in the presence or absence of provided structural variant calls. In some instances, such a method can have a higher power to detect causative variants in new cases than a non-independent method. In some instances,

In some instances, a method disclosed herein for example step (f) can comprise gene burden scoring. Gene burden scoring can comprise assigning a score to a gene based on its burden or the burden of the gene or of one or more variants of a gene. Gene burden scoring can be carried out by calculating the burden of variants for a gene based on the available data and mode of inheritance. In some cases, gene burden scoring can be carried out using the VAAST algorithm, as described herein.

In some instances, a method disclosed herein for example step (g) can comprise phenotype based gene prioritization. This step can have as an input one or more phenotypes of the subject. These phenotypes can be phenotypes associated with health or phenotypes associated with a disease or variant. The input phenotype(s) can be described in ICD codes in some cases. Phenotypes can be from any phenotype ontology, such as the Human Phenotype Ontology (HPO), or a gene function ontology. In some cases, the phenotype terms can be obtained by analysis of the Electronical Medical Record of the patient. In some cases, the phenotype terms can be obtained by Natural Language Processing of clinical notes from the Electronical Medical Record of the patient. In some cases, Phenotype based gene prioritization can comprise an algorithm that can rank a given set of genes relative to a subject phenotype. The algorithm can order genes, for example by the semantic similarity computed between phenotypic descriptors associated with each gene and those describing the subject. Phenotypic descriptor terms can be taken for example from the HPO, and semantic similarity can be derived from each term's information content. In some cases, the phenotype terms provided can be weighted by an assessment of their specificity, for example through large-scale analysis of Electronic Medical Records.

Phenotype based gene prioritization can be carried out by ontological propagation across a biomedical ontology graph relating phenotype terms among themselves with gene annotations based on prior knowledge. In some cases, such prioritization can be carried out using Bayesian networks utilizing a biomedical ontology graph relating phenotype terms among themselves with gene annotations based on prior knowledge.

Phenotype based gene prioritization can utilize data derived from mutation induced or observed in model organisms, and can correlate one or more genes to one or more phenotypes in such organisms to one or more orthologous genes and one or more orthologous phenotypes in humans. In some cases, such prioritization can utilize data derived from one or more mutations induced or observed in model organisms and can correlate one or more genes to one or more phenotypes in such organisms to one or more orthologous genes and one or more orthologous phenotypes in humans. In some such cases, the HPO ontology can be used as an intermediate.

In some instances, a method disclosed herein for example step (g) can be carried out by the PHEVOR algorithm, as described herein. In some instances, a method disclosed herein for example step (g) can be carried out by the Exomiser algorithm, which can be a tool which can find a potential disease causing variant from whole exome or whole genome sequencing dat. In some instances, a method disclosed herein for example step (g) can be carried out using the Phenolyzer algorithm, which can be a tool that can discover genes based on user-specific disease terms or phenotype terms. In some instances, a method disclosed herein for example step (g) can be carried out using the VarElect algorithm, which can be a next generation sequencing phenotyper which can rapidly prioritize variant genes based on a disease or phenotype of interest. In some instances, a method disclosed herein for example step (g) can be carried out using the OMIMexplorer algorithm, which can perform rapid integration of phenotype information with genotype information to assist with differential diagnostics of genetic disease, molecular variant prioritization, or novel gene-phenotype association discovery. In some instances, a method disclosed herein for example step (g) can be carried out by combining a similarity score to possible diseases derived from image analysis of patient's facial morphological features with variant impact

Phenotype based gene prioritization can be carried out by image analysis of one or more patient morphological features correlated with one or more phenotype ontologies or one or more gene lists. The ontologies and/or lists can be in some cases annotated to phenotype terms.

Image analysis can be of facial or other morphological features. Image analysis can be carried out by analyzing a photograph, a histological sample (e.g., a brighfield or fluorescent image), an MM image, a PET image, a CT image, an XRAY image, an ultrasound image, an OCT image, a bioluminescence image, or another image. Image analysis can comprise measurement or description of a length, width, area, color, quantity, texture, distribution, pattern, or other analysis of a feature of an image or a section of an image. In some cases, relevant genes identified using an image analysis technique can be learned from analysis of previously solved machine learning method or from crowd sourced diagnostics using a machine learning method. In some cases, genes identified using an image analysis technique be assessed for similarity to disease descriptions and/or corresponding relevant genes, wherein the genes can be learned from crowd sourced examples using machine learning methods.

In some instances, a method disclosed herein for example two steps (e.g., step (f) and step (g)) can be performed simultaneously. For example, step (f) may be performed first, or step (g) may be performed first, as they do not depend on each other.

In some cases, subsequent to step (g) or steps (f) and (g) performed simultaneously, the gene prioritization can be combined with the gene burden test. In some instances, a method disclosed herein for example step (h) may comprise the gene prioritization combined with the gene burden test. At this stage, pedigree information can be an input into the algorithm. Pedigree information can comprise phenotypic or genetic information about a family member, such as a parent.

In some instances, a method disclosed herein for example step (h) can be carried out by the PHEVOR algorithm, as described herein. In some instances, a method disclosed herein for example step (h) can be carried out by the Exomiser algorithm, which can be a tool which can find a potential disease causing variant from whole exome or whole genome sequencing dat. In some instances, a method disclosed herein for example step (h) can be carried out using the Phenolyzer algorithm, which can be a tool that can discover genes based on user-specific disease terms or phenotype terms. In some instances, a method disclosed herein for example step (h) can be carried out using the VarElect algorithm, which can be a next generation sequencing phenotype which can rapidly prioritize variant genes based on a disease or phenotype of interest. In some instances, a method disclosed herein for example step (h) can be carried out using the OMIMexplorer algorithm, which can perform rapid integration of phenotype information with genotype information to assist with differential diagnostics of genetic disease, molecular variant prioritization, or novel gene-phenotype association discovery. In some instances, a method disclosed herein for example step (h) can be carried out by combining a similarity score to possible diseases derived from image analysis of patient's facial morphological features with variant impact. In some instances, a method disclosed herein for example step (h) can be carried out using another phenotype based prioritization algorithm.

Combining phenotype-gene prioritization with a gene burden test can comprise calculating the probability of disease-involvement from data in the OMIM database, for each mode of inheritance described. In some cases, combining phenotype-gene prioritization with a gene burden test can comprise calculating the probability of disease-involvement from data in any gene-condition database such as Monarch31, Orphanet, ClinGen32, MONDO, MedGen, Panelapp, etc.

Next, one or more condition-gene databases can be queried for disease, mode of inheritance, and/or penetrance (i) and one or more clinical variant classification databases can be queried for matches with patient variants (j). In some cases, (i) and (j) can be performed simultaneously, (i) can be performed first, or (j) can be performed first. In some cases, either (i) or (j) or both may not be performed.

In some instances, a method disclosed herein for example (j) can be carried out by querying a database of variant of clinical relevance documenting their previously assessed pathogenicity. In some instances, a method disclosed herein for example, (j) can be carried out by querying the ClinVar database of clinical variants. In some instances, a method disclosed herein for example (j) can be carried out by querying the ClinVar database of clinical variants, considering committee expert review (when available), agreement between submissions, and the number and timeline of submissions. In some instances, a method disclosed herein for example (j) can be carried out by querying the HGMD database of clinically relevant variants. In some instances, a method disclosed herein for example (j) can be carried out by querying a database of clinically relevant variants and their pathogenicity assessment constructed from mining a medical literature corpus and analyzed automatically with Natural Language Processing methods to extract variant incidence and classification, with or without posterior manual curation. In some instances, a method disclosed herein for example (j) can be carried out by querying a database of variant pathogenicity assessments derived from high-throughput functional studies.

In some instances, a method disclosed herein for example (j) can comprise calculating a p-value of likelihood that a score calculated from the information of any one or more of steps (e), (f), (g), (h), (i), or (j) may occur by random chance.

In some instances, a Bayes factor can be calculated (k). The Bayes factor can be calculated to maximize among possible models for each gene with a variant of impact. In some cases, a final output sorting one or more genes of significance can be provided. Such an output can be used for example for clinical review or for research purposes. Calculation of a Bayes factor is described in more detail below.

In some instances, the Bayes factor calculation in (k) can be replaced by calculation of the Akaike information criterion, p-values from hypothesis testing, one or more scores from one or more machine learning classifier, or another acceptable method.

In some instances, a method disclosed herein for example k can be carried out by using a machine learning algorithm. Such a machine learning algorithm can be trained for example using a database of previous cases, which can be analyzed manually by clinicians, and/or cross validated with a set second set of withheld cases. Such a machine learning algorithm can comprise one or more of naïve Bayes, Bayesian networks, support vectors machines, random forests, decision trees, gradient boosted trees, or deep learning.

In some instances, in a method disclosed herein for example after the calculation in (k), all variants and genes can be listed. In some cases, after step (k), only variants and genes for which the Bayes factor is significant are provided.

In some instances, a method disclosed herein for example the calculation in (k) can be carried out by sorting genes by a p-value. In such cases, each variant may be listed. In some such cases, only variants past a specified significance value may be listed. Sample significance values which may be used include but are not limited to p<0.1, p<0.05, p<0.01, p<0.005, p<0.001, p<0.0005, or p<0.0001. In some cases, such sorting can be performed by a machine learning algorithm.

In some instances, a method disclosed herein for example the calculation in (k) can comprise direct physical phasing of variants when a multiplicity of them are present in a gene, which can be obtained during variant calling.

In some instances, a method disclosed herein for example the calculation in (k) can comprise assessing each of one or more variants for their likelihood of being artifacts based on previous knowledge of such artifacts in data from the different sequencing platforms. Such information can be provided as additional input, for example in (k).

In some instances, a method disclosed herein for example the calculation in (k) can comprise assessing each of one or more variants for their likelihood of being artifacts based on previous knowledge of such artifacts in data from the Illumina platform. Such information is provided as additional input, for example in (k).

In some instances, a method disclosed herein for example the calculation in (k) can comprise assessing each of one or more variants for their likelihood of being artifacts based on previous knowledge of such artifacts. Previous knowledge can be for example in data, such as that from the Ion Torrent platform 33. Such information is provided as additional input, for example in (k).

In some instances, a method disclosed herein for example the calculation in (k) can comprise assessing each of one or more variants for their likelihood of being artifacts based on previous knowledge of such artifacts in data, for example from the BGI platform. Such information is provided as additional input for example in (k).

In some instances, a method disclosed herein for example the calculation in (k) can comprise assessing each of one or more variants for their likelihood of being artifacts based on previous knowledge of such artifacts, for example in data from the SOLiD platform 34, Such information is provided as additional input, for example in (k).

In some instances, a method disclosed herein for example the calculation in (k) can comprise assessing each of one or more variants for their likelihood of being artifacts based on previous knowledge of such artifacts in data from the Oxford Nanopore platform, and this information is provided as additional input, for example in (k).

In some instances, a method disclosed herein for example the calculation in (k) can comprise assessing each of one or more variants for their likelihood of being artifacts based on previous knowledge of such artifacts in data from the Pacific Biosciences platform, and this information is provided as additional input, for example in (k).

In some instances, a method disclosed herein for example the calculation in (k) can comprise assessing each of one or more variants their likelihood of being artifacts based on previous knowledge of such artifacts in data from sequencing by synthesis platforms, and this information is provided as additional input, for example in (k).

In some instances, a method disclosed herein for example the calculation in (k) can comprise assessing each of one or more variants for their likelihood of being artifacts based on previous knowledge of such artifacts in data from sequencing by ligation platforms, and this information is provided as additional input, for example in (k).

In some instances, a method disclosed herein for example the calculation in (k) can comprise assessing each of one or more variants for their likelihood of being artifacts based on previous knowledge of such artifacts in data from sequencing by single molecule sequencing platforms, and this information is provided as additional input, for example in (k).

In some instances, a method disclosed herein for example the calculation in (k) can comprise assessing each of one or more variants for their likelihood of being artifacts based on previous knowledge of such artifacts in data from the Illumina platform, and this information is provided as additional input, for example in (k).

In some cases, a quality value for each variant can be calculated. Such a calculation can comprise analysis of the distribution of one or more reads harboring a plurality of variants up to each variant in the genotype, and in some cases considering the chromosome location and the sex of the patient. In some instances, a method disclosed herein for example (k) can comprise identifying the sex of the patient from the sequencing data.

In some cases, the ancestry of the patient can be inferred from the sequencing data to adjust the probability calculated in (e), for example when the method used for deleteriousness assessment used as input population frequency data from internal or public allele frequency databases such as gnomAD. In some cases, the ancestry of the patient can be inferred from the sequencing data. In some cases, this inference can be used to adjust the probability calculated in step (e).

In some cases, the method can comprise identifying if the subject carries one or more segments of homozygosity, which can result in consanguinity. In some such cases, the method can further comprise labeling variants harbored in such segments. Such a method can further comprise adjusting the prior probability that such variant is disease causing in a method disclosed herein for example step (k).

In some cases, the phenotypes described in the patient are correlated to genes based on prior knowledge and/or biomedical ontologies. Such cases can further comprise obtaining and then evidence for their expression in tissues compatible with such phenotypes from a database, such as one or more proprietary or public tissue-expression database. In some cases, prioritization can be adjusted in the calculations in a method disclosed herein for example of step (k).

In some instances, the phenotypes described in the patient are correlated to genes based on prior knowledge and/or biomedical ontologies. Subsequently, an evidence for their expression in tissues compatible with such phenotypes may be obtained from the GTex project 35 database, and depending on their expression prior probability may be adjusted in the calculations of step (k).

In some cases, a clinician can review a report outputted in a method disclosed herein for example (l). In some cases, a clinician can make a diagnostic decision. The diagnostic decision can be a final decision or it can be a decision that can narrow down a differential diagnosis. In some cases, the decision can be made using additional data. In some cases, the decision can be optional.

Steps (d) through (l) can be performed quickly. In some cases, steps (d) through (l) can be completed in about 30 minutes, about 1 hour, about 1.5 hours, about 2 hours, about 3 hours, about 6 hours, or about 12 hours. In some cases, steps (d) through (1) can be completed in less than about 30 minutes, less than about 1 hour, less than about 1.5 hours, less than about 2 hours, less than about 3 hours, less than about 6 hours, or less than about 12 hours.

In some cases, a treating physician can receive a diagnostics report (m). Such a diagnostics report can be based on the diagnostics decision of (l). In some cases, a treating physician can begin, adjust, or stop management of a disease or a therapy based on the contents of the diagnostics report. In some cases, (m) can be optional.

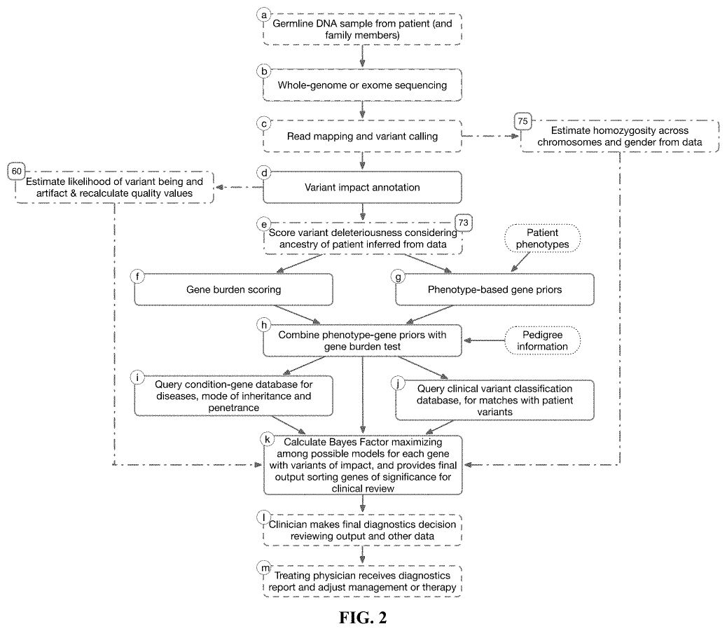

In some cases, an extended version of the method can be implemented. An example of such an extended method is illustrated in FIG. 2.

In some cases, an extended method can comprise estimation of homozygosity across chromosomes and/or gender from the provided data. Such a step can use an output from the read mapping and variant calling step as an input.

In some cases, an extended method can comprise estimation of the likelihood that a variant is an artifact using an output from (d). In some cases, quality values (including the likelihood that a variant is an artifact) can be recalculated. In some cases, artifacts can be removed during this step. Such a step can be repeated iteratively. In some cases, such a step can be repeated until the data is sufficiently free of artifacts. The output of this step can serve as an input to (k).

In some cases, an extended method can comprise amending € such that the scoring of variant deleteriousness considers ancestry of the patient inferred from data. Such data can be from a survey, from a patient history, from an Electronic medical record, from a family history, from a database, or from sequencing data.

### Structural Variance Inference and Vetting

A barrier to using structural variants for the diagnosis if rare genetic disease can be a high false positive rate associated with bioinformatic tools for structural variant identification using short-read sequencing techniques. In some instances, a structural variance inference and vetting method provided herein can overcome structural variant calling noise, and can combine noise with phenotype data for the diagnosis of mendelian conditions. A noise can comprise a signal to noise ration or a baseline noise. The signal to noise ratio using the method described herein can be improved by about: 10%, 20%, 30%, 40%, 50%, 60%, 70%, 80%, 90%, 99%, or a percentage amount in between any two percentage amounts mentioned hereinbefore. Methods can be applied to diagnosis of rare genetic disease or common, complex disease. In some instances, methods can effective discover and employ structural variants for mendelian condition diagnosis with high accuracy.

In some instances, a structural variance inference and vetting method can be incorporated into methods provided herein, for example at step (e). In some instances, a structural variance inference and vetting method can be used independently, for example as a filter for structural variant calls provided by upstream structural variant identification algorithms, or in tandem, for example to downgrade or filter out low quality structural variant calls.

A structural variance inference and vetting method can directly infer one or more medium to large sized structural variant events directly from the data associated with small variant calls (e.g., SNVs, small indels) readily available in variant call format (VCF) files, which can be routinely produced during secondary analysis of next-generation sequencing data, for example from whole-exome or whole-genome assays. Methods can be validated for example with VCFs from paired-end sequencing data or another platform that can produce VCFs or similar data.

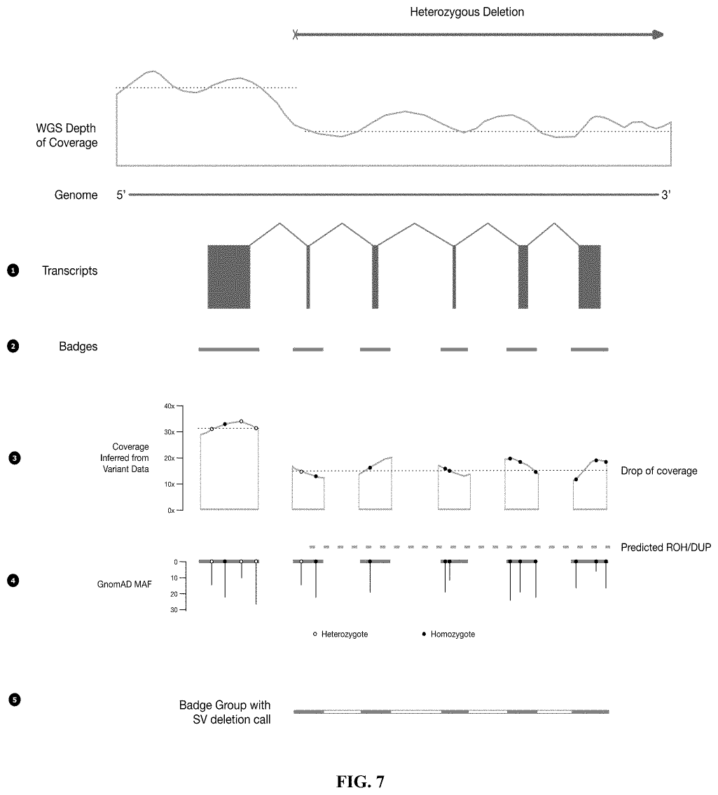

An example of a method for structural variance inference and vetting is outlined in FIG. 7. As shown in FIG. 7 a first step can comprise obtaining a list of transcripts for genes. Such a list of transcripts can comprise all transcripts, or a representative transcript selected for each gene (e.g., a canonical transcript). Transcripts can be mapped to their genomic locations. For example, a list of transcripts for all Human genes can be obtained, from which either all, or a representative transcript for each gene is selected (canonical transcript).

In some instances, for a transcript, a set of badges that can represent the DNA sequence coordinate can be constructed. The badges may span each transcript exon region in the list of transcripts. The badges may also comprise an upstream and a downstream buffer region to capture splice regions. Such buffer regions can be for example about 1500 bases to 2500 bases such as 2 kilobases (kb), or less than: about 1000 bases, about 1500 bases, about 2000 bases, about 2500 bases, about 3000 bases, or at least: about 1000 bases, about 1500 bases, about 2000 bases, about 2500 bases, about 3000 bases, or a range between any two foregoing values. Such badges can be coordinated for a plurality of transcripts, or for each transcript.

In some instances, a set of variants for the genome or whole exome of a patient or subject being considered for diagnostics can be obtained, e.g., as a VCF. Variants that overlap one or more badges can be identified. Variants can then be analyzed for evidence of deletions or duplications, for example by analysis of the read depth reported for such variants as compared to the distribution of coverage for all variants in the genome or exome. In some instances, a badge can present a higher or lower average depth of coverage reported by the variants called. In some instances, a badge can be a candidate for a duplication event if it presents a higher than average depth of coverage. In some instances, a badge can be a candidate for a deletion event if it presents a lower than average depth of coverage.

Informed with a population allele frequency for a patient's or subject's variants present in the badges (obtained e.g., from global databases of allele frequencies such as gnomAD), the method can identify whether successions of homozygote calls present in a set of adjacent badges of a transcript are in excess of what would be expected given the population allele frequencies of such variants. In some instances, the presence of runs of homozygosity can be evidence for each variant for evidence of a succession of positions with imbalance of alleles, which can be possibly due to duplication of one or more haplotype. In some instances, an absence of common variants can be used to infer homozygous deletions.

The ploidy of each badge given the data collected can be estimated. Estimation can be performed for example using a Bayesian approach, or another acceptable approach. In some instances, adjacent badges exhibiting a same ploidy can be combined into a badge group. In some instances, badges with ploidy greater or less than 2 for autosomes (e.g., 1, or 3, 4, 5, 6, 7, 8, 9, or 10 or greater), 2 for the X chromosomes in females, and/or less than or more than 1 for the X chromosome in males can be reported as a structural variant.

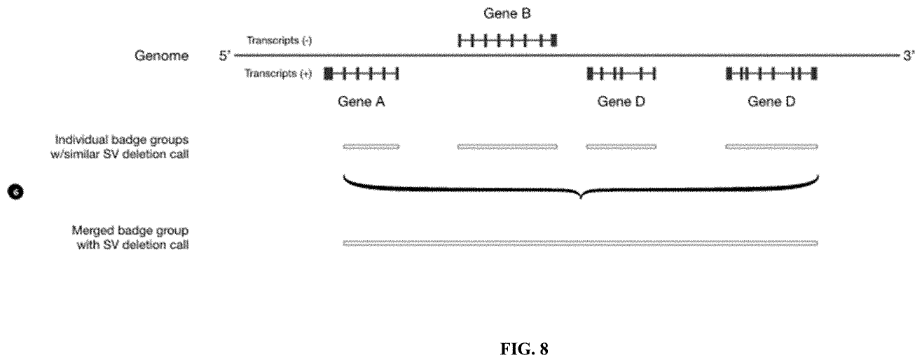

In some instances, adjacent badge groups of anomalous matching ploidy can be combined to form a larger badge group. In some instances, the larger badge group can represent a larger structural variant (FIG. 8). In some instances, precise genomic boundaries of identified structural variant events cannot be identified. In some instances, badge groups can be assessed for pathogenicity. Once structural variant events (e.g., represented as badge groups) are inferred, the structural variant events can be associated with one or more disease phenotypes. These structural variant events can be assessed for pathogenicity. Assessment can be carried out for example by an extension to a method for near-instant interpretation and disease gene finding for rare disease from genomes and exomes.

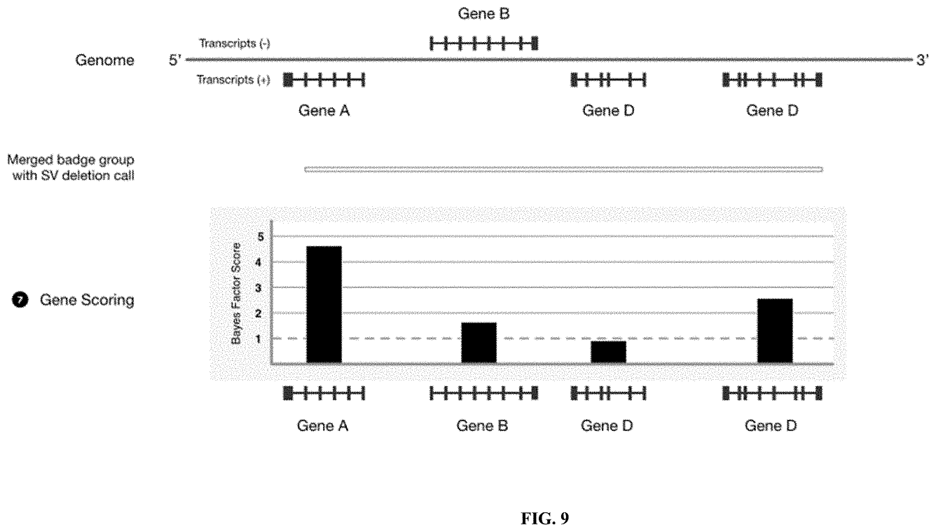

In some instances, badge groups can be scored for pathogenicity by merits of the genes harbored by the event (FIG. 9). A score such as a Bayes factor score can be calculated for each gene in the badge group. Bayes factor calculation can be carried out for example as described hereinbelow. In some instances, an acceptable alternative to a Bayes factor score can be calculated.

In some instances, for example, computation of a Bayes factor can comprise computation of a severity score as the reciprocal of the ploidy of the badge. Other methods may also be used. In some instances, for example, whether a gene has been identified as dosage sensitive in literature or in curated databases of dosage sensitivity can be considered. Considering the direction of the ploidy (e.g., heterozygous deletion vs. triplication), the severity score can be increased for one or more genes in the badge with such attributes. For example, a severity score can be differently increased for genes having haploinsufficiency vs. gain of function. Such deleteriousness assessments can replace a VVP score used for small variants in some instances.

In some cases, if any genes resent significant evidence of causality, for example as defined by a Bayes factor threshold, the badge(s) can be reported as candidate causative variant(s) as promoted by their harboring gene with high Bayes factor. In some instances, the Bayes factor can also be used to differentiate between incidental genes hitchhiking in the structural variants vs. real causal genes involved in the disease. In some instances, such a feature can provide insight in the causality of a large structural variant, rather than simply reporting a diagnosis based on a database lookup using structural variant begin and end coordinates. In some instances, component scores used as an input to a Bayes factor calculation or other acceptable calculation can inform a clinician or other user about candidacy of the structural variant as a causal pathogenic variant.

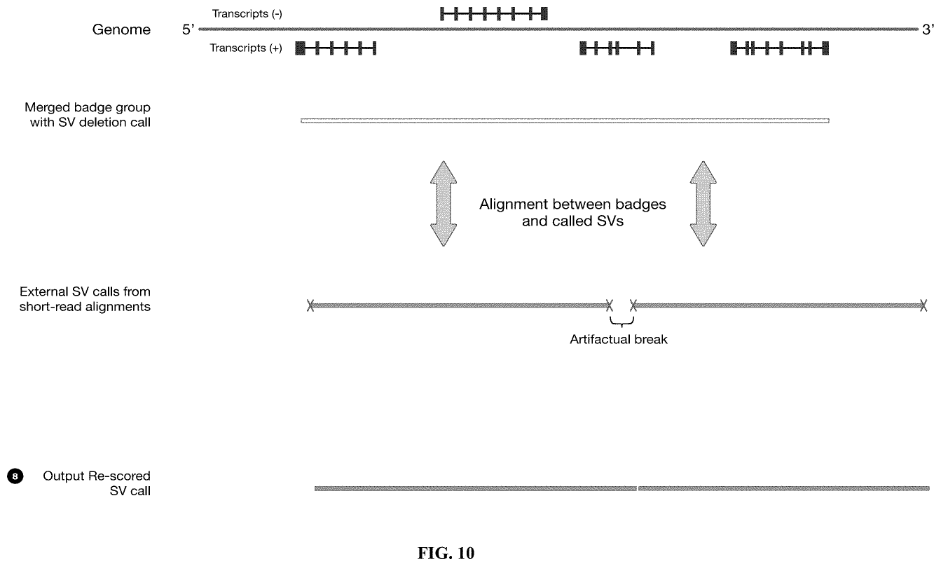

In some instances, a badge group can be reported as an inferred structural variant. In some cases, this reporting may not require orthogonal conformation, for example if a physical structural variant is not derived directly from read alignments. In some instances, if structural variant calls have been provided in parallel from the read alignments, these two forms of evidence can be aligned and contrasted (FIG. 10). In some cases, this can be a form of variant rescoring, wherein external structural variant calls can gain confidence if one or more corresponding badge groups are identified from the variant data.

In some instances, artifacts that may break structural variant calls inferred from read alignments and breakpoints can be reduced or eliminated when alignment is based on inference using badges. In some instances, a corresponding structural variant call can be prioritized for diagnostic purposes based on phenotype priors (e.g., determined using Phevor) of the harbored genes with higher confidence than by inferring a structural variant using another method. Such increase in confidence can reduce the need for orthogonal confirmation. Based on the method described herein, c the SV inference can be leveraged in combination with the prioritization based on: variant deleteriousness, genes derived from patient phenotypes and mining the HPO graph (our Phevor algorithm), data from sequence and genome evaluation databases (e.g. OMIM, CLinVar), the number of false positive in SV prediction can be reduced and a positive predictive value (PPV) can be increased. The PPV may include the SVs that are causal to a disease or highly associated with the disease. Based on the results shown herein, a positive predictive value in one of the experiment (example 4) was at 100%. The method described herein can achieve a positive predictive value of about 90% to about 100%. The achieved PPV may be between about 95% to about 100%, about 90% to about 95%, about 97% to about 100%, about 98% to about 100%, about 94% to about 98%. The achieved PPV may be about 94%, about 95%, about 96%, about 97%, about 98%, about 99%. The method described herein may lead to a false positive rate of at most about: 10%, 9%, 8%, 7%, 8%, 5%, 4%, 3%, 2%, 1%, or less. The false positive rate of the method for inferring a structure variant as described herein may be between about 0% to about 1%, about 0% to about 3%, about 0% to about 5%, about 1% to about 5%, or 5% to about 10%. The sensitivity of the method described here in predicting a disease causing structural variant can be about 90%, about 92%, about 95%, about 97%, about 100%, or a percentage amount between any two percentage amounts mentioned hereinbefore.

In some cases, a disease may be treated based solely on symptoms of a patient (or subject); determining a genetic cause of a disease using the methods described herein may affect the treatment of the patient (or subject). In some cases, a subject may have a genetic predisposition of a disease and detecting a genetic variant that may be associated with a disease with a high sensitivity achieved using the method described herein can be leveraged to prevent the disease or treat a subject for the disease before the disease becomes symptomatic.

### Bayes Factor Calculation

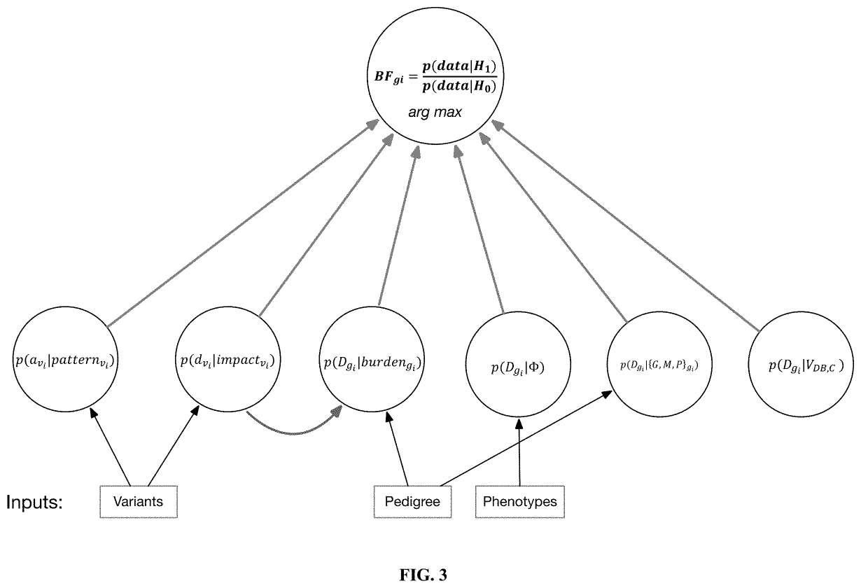

FIG. 3 illustrates a sample method for how the calculation in (k) can be implemented as a Bayes factor, and the data and other probabilities that can be used for the Bayes factor calculation. In some cases, a possible generic implementation of the method can be carried out by calculating a Bayes factor as the score used to prioritize genes and which can be used as a cut-off to remove from consideration unrelated genes (commonly, a log₁₀ Bayes factor less than 1.0 implies insufficient evidence for the hypothesis). FIG. 3 depicts a conceptual diagram of the Bayes Factor calculation and the information used. In the lower tier of the figure the most relevant inputs are indicated.

This method can calculate a score that accumulates the evidence in favor that a gene, and specific variants harbored in such gene, in terms of the probability that they are the cause of the disease of the patient. The method can also consider information on whether a gene with sufficient evidence has been previously described as associated with a rare genetic disease compatible with the phenotype of the patient, and whether the mode of inheritance observed in the patient is compatible to this prior knowledge.

Such a method can be applied to single cases, parent-offspring trios, or other types of pedigrees; having family members permits more rigorous evaluation of the putative mode of inheritance, and down-scores irrelevant variants, resulting in higher discrimination. Such a score can be used a cut-off, for example to only show genes and variants that are likely to be relevant for the case (commonly, a log₁₀ Bayes factor less than 1.0 implies little support for the hypothesis). Such a method can also highlight genes and variants previously deemed pathogenic by other clinicians, but which can be unlikely or not optimally likely to be the primary cause of the patient's disease (e.g. because mode of inheritance may be incompatible with prior knowledge, e.g. the variant is heterozygous, but the gene it resides may be associated with recessive disease), and therefore can be described (and reported) as incidental findings.

The formula below can be sued as a generic description of how a Bayes factor can be calculated (see en.wikipedia.org/wikiBayes_factor).

$$B {F}_{g i} = \frac{p ( data \vert {H}_{1} )}{p ( data \vert {H}_{0} )}$$

The Bayes factor can test the evidence in favor for the gene i as disease causing over the null hypothesis of no causality. For example, in FIG. 3, “arg max” indicates that the max p(data|H) is identified for each variant-gene combination by considering all alternatives for each parameter of the model, e.g. mode of inheritance and other parameters.

In FIG. 3, each circle can represent an input to the Bayes factor (or other score used to implement the method) in its calculation. These inputs can include: (I) Probability that a variant i is an artifact (a), given a pattern observed in the genome for this variant (optional):

p(aᵥ_{ᵢ}|patternᵥ_{ᵢ})

(II) Probability that a variant i is deleterious (d), given their predicted impact using a deleteriousness score:

p(dᵥ_{ᵢ}|impactᵥ_{ᵢ})

(III) Probability that a gene i is involved in the patient's disease D, given the burden of damaging variants harbored in that gene:

p(D_{g}_{ᵢ}|burden_{g}_{ᵢ})

(IV) Probability that a gene i is involved in the patient's disease D, given the aggregated phenotypes of the patient and their relationship and annotations in a phenotype or functional ontology (Φ):

p(D_{g}_{ᵢ}|Φ)

(V) Probability that a disease gene i can influence the patient's Disease phenotype D, given a database of gene-conditions documenting known disease genes (G), compatible modes of inheritance (M), and penetrance of the disease (P):

p(D_{g}_{ᵢ}|{G,M,P}_{g}_{ᵢ})

Or (VI) Probability that a variant(s) harbored in gene i, is (are) pathogenic and thus affect the patient's disease D, given entries in a database of classified variants contributed by clinical labs (VDB), considering quality and history of submissions C:

p(D_{g}_{ᵢ}|V_{DB,C})

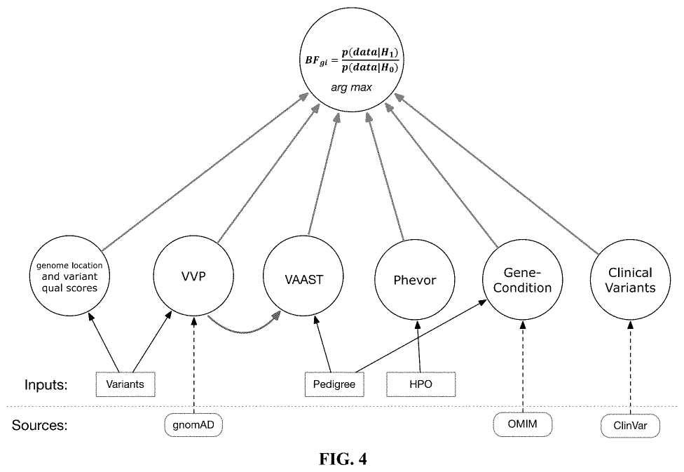

FIG. 4 illustrates a specific implementation of key characteristics of the method described herein. FIG. 4 shows that in I, a variant quality score can be recalculated from the count of reads harboring each of the allele of a variant, considering whether they reside in an autosome or a sex chromosome and the gender of the patient (inferred from the data). In addition, the score can reflect the probability of variant calling artifacts arising at each position of the genome when the Illumina sequencing technology is employed, trained in a large dataset of whole-genome and whole-exome sequencing data. For II, the VAAST Variant Prioritized score can be used; for III, the VAAST algorithm can be used; for IV the Phevor algorithm can be used; for V, data extracted from the OMIM database can be used; and for VI, data extracted from the ClinVar database can be used. These methods can be then combined to manufacture the input for the Bayes factor calculations.

The computation of the Bayes factor can be short. In some cases, the computation can be performed in not more than 1 second, not more than 2 seconds, not more than 3 seconds, not more than 5 seconds, not more than 10 seconds, not more than 15 seconds, not more than 30 seconds, not more than 45 seconds, or not more than 60 seconds. In some cases, the computation can be performed in about 1 second, about 2 seconds, about 3 seconds, about 4 seconds, about 5 seconds, about 10 seconds, about 15 seconds, about 30 seconds, about 45 seconds, or about 60 seconds. In some cases, the computation can be performed in between 1 second and 60 seconds, between 5 seconds and 60 seconds, between 10 seconds and 60 seconds, between 15 seconds and 60 seconds, between 30 seconds and 60 seconds, between 45 seconds and 60 seconds, between 1 second and 45 seconds, between 5 seconds and 45 seconds, between 10 seconds and 45 seconds, between 15 seconds and 45 seconds, between 30 seconds and 45 seconds, between 1 second and 30 seconds, between 5 seconds and 30 seconds, between 10 seconds and 30 seconds, between 15 seconds and 30 seconds, between 1 second and 15 seconds, between 5 seconds and 15 seconds, between 10 seconds and 15 seconds, between 1 second and 10 seconds, between 5 seconds and 10 seconds, or between 1 second and 5 seconds.

### Output

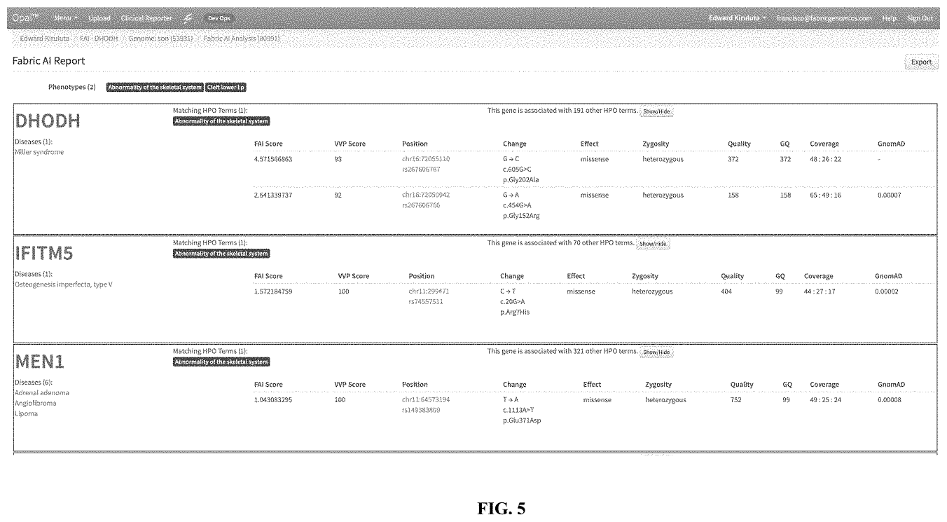

In some cases, the output that can be parsed and rendered in a clinician friendly report, together with annotations and link-outs to original clinical data sources, for example as shown in FIG. 5. This data can be used by a clinician or a researcher to decide if the recommended short list of possible disease-causing genes and variant is a believable candidate disease variant(s). At that point, the clinician or researcher can promote one or more of these variants to a clinical report, for example finishing the disease-gene curation, classifying the variants using professional guidelines such as the ACMG/AMP classification 21, or writing a description for the reported variants.

### Computer Systems

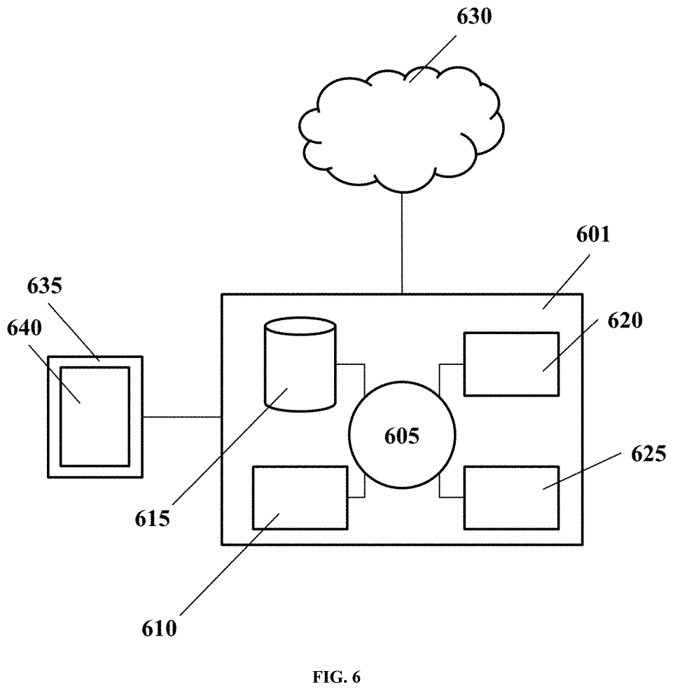

The present disclosure provides computer systems that are programmed to implement methods of the disclosure. FIG. 6 shows a computer system 601 that is programmed or otherwise configured to perform methods described herein. In some cases, the computer system can be configured to (a) provide a set of genes having variants, which set of genes is generated from a nucleic acid sample of said subject; (b) process said set of genes against a known set of genes to identify at least one gene of said set of genes having a variant, wherein said known set of genes have known variants associated with a set of phenotype, and wherein said variant is associated with a phenotype of said set of phenotypes; (c) calculate a likelihood that said gene identified in (b) is causative of said phenotype; and (d) output a report that is indicative of said at least one gene having said variant identified in (b) and said likelihood calculated in (c). The computer system 601 can regulate various aspects of disclosure, such as, for example, accessing databases, performing sequencing, read mapping, variant calling, variant impact annotation, scoring variant deleteriousness, gene burden scoring, phenotype based gene prioritization, combining phenotype-gene prioritization with a gene burden test, querying a condition-gene database, querying a clinical variant classification database, calculating a Bayes factor or performing another calculation for example to maximize among possible models for each gene with variants of impact, or providing an output or report such as one that sorts genes of significance for clinical review. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, instructions, input for patient or pedigree information including phenotypes, database selection, algorithm selection, a report viewer, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, (a) process a set of genes having variants, which set of genes is generated from a nucleic acid sample of said subject; (b) process said set of genes against a known set of genes to identify at least one gene of said set of genes having a variant, wherein said known set of genes have known variants associated with a set of phenotype, and wherein said variant is associated with a phenotype of said set of phenotypes; (c) calculate a likelihood that said gene identified in (b) is causative of said phenotype; and (d) output a report that is indicative of said at least one gene having said variant identified in (b) and said likelihood calculated in (c).

### Example 1

Causative variants were systematically spiked into a number of control genomes for a variety of disease and modes of inheritance, allele frequencies, and penetrance, and were analyzed. In addition, dozens of previously solved clinical cases were retrospectively analyzed. The method was able to quickly identify disease genes and variants responsible for the patient's phenotype with significantly greater precision than VAAST and PHEVOR; for over 80% of the clinical test cases comprising parent-offspring trios the output includes the correct gene as the stop scoring gene, while in the remaining cases the correct gene is among 2-3 candidates offered. The method still retains the ability of VAAST and PHEVOR to identify novel disease genes, but significantly reduces the time spent on reviewing candidate causative genes and variants.

### Example 2

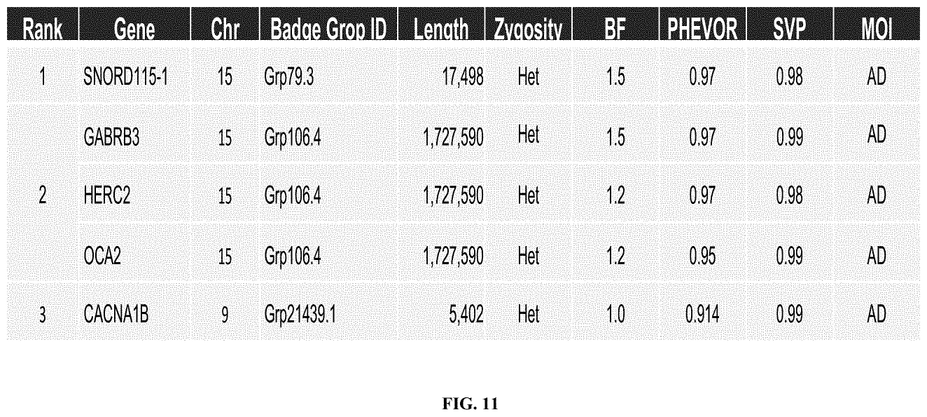

An example of a case of a patient previously diagnosed as having Prader-Willi Syndrome is shown in FIG. 11. Prader-Willi Syndrome can be caused by large structural variants on Chromosome 15. A method for inferring structural variants using badges was performed on the genetic information of the patient. In addition, Phevor analysis was performed on the data. The Phevor column reports the gene phenotype prior from patient's phenotype, and the SVP column is the deleteriousness score based upon the likelihood of seeing the observed number of homozygous SNVs and small indels within the region of the badge. The SVP and Phevor values are posterior probabilities, with values near one suggestive of causality. MOI is mode of inheritance, which in this case is autosomal dominant (Haplo insufficient). Scientific literature reports have previously implicated the genes GARBRB3, HERC2, and OCA2 as potentially causal for the disease. Many other genes overlapped by the Badge Groups did not achieve a significant Bayes Factor, showcasing how the method can provide insights in causality without literature input. Furthermore, although the method identifies 2 badge groups in Chromosome 15 that it believes are involved with the patient's disease, both badges are likely pieces of the same singular event. In this analysis, the VCF file did not contain any independent SV calls, and the hit was thus obtained by our inference method exclusively, showing the utility of the method for analysis old cases with no SV calls.

### Example 3

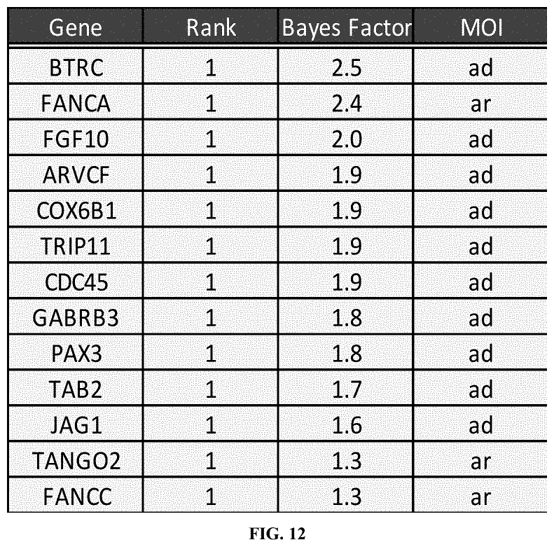

Thirteen cases with previously reported causal structural variants from the Rady Children's Hospital NICU, rapid WGS program were reanalyzed using a method for inferring structural variants using badges as described above. The results of this analysis are shown in the table in FIG. 12. In all cases, the causal structural variant was identified by the method, with significant Bayes factors. Based on the results shown herein, a positive predictive value in this experiment was at 100%.

While representative embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

1.-42. (canceled)

43. A computer-implemented method for inferring a structural variant, said method comprising:
(a) constructing a set of badges to span an exon, an upstream region, a downstream region, a gene regulatory element, or any combination thereof of one or more genes or transcripts thereof;
(b) identifying one or more genetic variants having one or more attributes to a human subject and overlapping said set of badges, and using said one or more attributes to determine a ploidy of said one or more genetic variants in said set of badges; and
(c) reporting a structural variant by using at least said ploidy determined in (b) to indicate a change in gene or regulatory element dosage caused by said structural variant that deviates from a normal human karyotype.

44. The method of claim 43, wherein said ploidy is determined based at least in part on an analysis of (i) discrepancy in distributing genetic variant alleles zygosities from expectation for a genomic segment overlapped by said badges or (ii) a distribution of reads harboring alternative alleles for said one or more genetic variants overlapped by said badges.

45. The method of claim 43, wherein said one or more genetic variants are from a whole-genome sequencing or whole-exome sequencing of a human subject.

46. The method of claim 43, wherein said badges are configured to span (i) an exon region of a transcript of a human genome or (ii) each exon of said transcripts.

47. The method of claim 43, wherein said badges further comprise (i) an upstream or a downstream buffer region associated with said exon region of said transcript or (ii) a gene regulatory region.

48. The method of claim 43, wherein said badges represent a DNA sequence coordinate of said exon region of said transcript.

49. The method of claim 43, wherein said ploidy is determined based at least in part on (i) a frequency of its alleles in human populations of said one or more genetic variants or (ii) defining an expectation of zygosity of overlapping genetic variants from a subject by comparing to a frequency of its alleles in human populations of said one or more genetic variants.

50. The method of claim 43, wherein said structural variant is associated with a disease phenotype or a gene associated with a disease phenotype.

51. The method of claim 43, further comprising determining a score inferring severity of an impact of a structural variant on a structure of overlapping genes that can be associated with a disease phenotype.

52. The method of claim 43, wherein said genetic variants or said genes or transcripts thereof have been ranked by VAAST, VVP, PHEVOR, pVAAST, SIFT, CAD, ANNOVAR, a burden-test, a sequence conservation scoring method, a machine learning method, or any combination thereof.

53. The method of claim 43, further comprising prioritizing a single nucleotide variant (SNV), an insertion or deletion (INDEL), or said structural variant.

54. The method of claim 43, further comprising automatically prioritizing compound heterozygous genotypes comprising a single nucleotide variant (SNV) or an insertion or deletion (INDEL) in trans to a larger structural variant.

55. The method of claim 43, further comprising merging said badges if adjacent in genome coordinates, discrepant to expectation of said human normal karyotype, or are of similar inferred ploidy.

56. A method for inferring a structural variant, said method comprising:
(a) providing one or more genetic variants attributable to a subject;
(b) identifying said one or more genetic variants overlapping one or more badges, wherein said one or more badges represents a portion of a human genome;
(c) determining a ploidy of said one or more genetic variants based at least in part on an analysis of sequencing read depth of said one or more genetic variants; and
(d) reporting a structural variant by using at least said ploidy determined in (b) to indicate a change in gene or regulatory element dosage caused by said structural variant that deviates from a normal human karyotype.

57. A method for analyzing a genome of a subject, comprising:
(a) providing a set of genes or gene regulatory elements suspected of having one or more single nucleotide variants (SNVs), insertions or deletions (INDELs), structural variants, or any combination thereof, which set of genes or gene regulatory elements is generated from a nucleic acid sample of said subject;
(b) processing said set of genes or gene regulatory elements against a known set of genes or gene regulatory elements to identify at least one gene or gene regulatory element of said set of genes or gene regulatory elements that is associated with a known set of phenotypes, wherein said known set of phenotypes is associated with one or more phenotypes of said subject;
(c) determining a likelihood that said at least one gene or gene regulatory element identified in (b) is causative of a phenotype; and
(d) outputting a report that is indicative of said at least one gene or gene regulatory element identified in (b) and said likelihood determined in (c).

58. The method of claim 57, wherein said set of genes or gene regulatory elements comprises genetic variants.

59. The method of claim 57, wherein (a) further comprises sequencing a DNA sample of said subject.

60. The method of claim 57, wherein (b) further comprises annotating variant impact on overlapping gene structure or regulatory element locations.

61. The method of claim 57, wherein (b) further comprises determining score variant deleteriousness on gene function.

62. The method of claim 57, wherein (b) further comprises gene burden scoring.

63. The method of claim 57, wherein (b) further comprises phenotype-based prioritizing.

64. The method of claim 57, wherein said one or more phenotypes of said subject is the same as one or more phenotypes in said known set of phenotypes.

65. The method of claim 57, wherein said at least one gene or gene regulatory element identified in (b) is causative of a disease, and wherein said disease is associated with said known set of phenotypes.

66. The method of claim 57, wherein (c) further comprises determining a Bayes factor.

67. The method of claim 57, wherein (c) further comprises using a machine learning algorithm.

68. The method of claim 57, wherein (c) further comprises integrating variant, pedigree, phenotype information, or any combination thereof about said subject.

69. The method of claim 57, wherein (c) further comprises using ExAC, gnomAD, OMIM, ClinVar, GARD, Orphanet, HGMD, or any combination thereof as an input source.

70. The method of claim 57, wherein said INDEL is less than 50 bp, or said structural variant is 50 bp or greater than 50 bp.
